(19) United States
(12) Patent Application Publication
(10) Pub. No.: US 2004/0157738 A1
(43) Pub. Date: Aug. 12, 2004

(54) Novel oxygen containing fused cyclic derivatives and herbicidal, desiccant and defoliate compositions containing them

(75) Inventors: Masamitsu Tsukamoto, Kusatsu-shi (JP); Hiroshi Kikugawa, Kusatsu-shi (JP); Makiko Sano, Kusatsu-shi (JP)

Correspondence Address: SUGHRUE MION, PLLC, 2100 Pennsylvania Avenue, NW, Washington, DC, 20037-3213, US

(73) Assignee: ISHIHARA SANGYO KAISHA, LTD.

(21) Appl. No.: 10/364,338

(22) Filed: Feb. 12, 2003

(51) Int. Cl. A01N 43/84; A01N 43/42; C07D 265/36; C07D 215/36

(52) U.S. Cl. 504/224; 504/247; 544/105; 546/153

(57) ABSTRACT

Oxygen containing fused cyclic compounds of the formula (I):

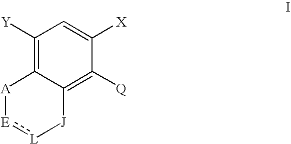

or their salts, process for producing them and their use as herbicides, plant growth regulators, desiccants and defoliants. The variables of formula (I) are defined in the specification.

### BACKGROUND OF THE INVENTION

1. Field of the Invention

The present invention relates to novel oxygen containing fused cyclic derivatives or their salts, a process for producing them, and their use as herbicides, plant growth regulators, desiccants or defoliants.

2. Description of the Related Art

JP 09301973 discloses the herbicidal utility of chromene derivatives, in which the phenyl ring of the described compounds was substituted with a hetero-ring such as uracil. Recently WO 01/10861 also disclosed chromene derivatives which have potent herbicidal activity in pre-emergence and post-emergence applications.

EP 271170 and U.S. Pat. No. 4,881,967 described chroman derivatives, in which the phenyl ring of the described compounds was substituted with many kinds of hetero-rings including phthalamide and triazolinone. WO 02/24704 disclosed chroman derivatives having herbicidal properties with a hetero-ring such as tetrahydro-6-oxa-2,3a,8a-triaza-azulene-1,3-dione.

Despite the broad coverage of these patents, the specific oxygen containing fused cyclic compounds of the formula I mentioned below are novel and can be used to effectively control a variety of broad or grassy leaf plant species.

### SUMMARY OF THE INVENTION

The present invention delineates a method for the control of undesired vegetation in a plantation crop by the application to the locus of the crop an effective amount of a compound described herein. The present invention provides certain herbicidal oxygen containing fused cyclic derivatives of the formula I including all geometric, tautomeric and stereo isomers, and their salts, as well as compositions containing them, methods of preparation for these compounds and intermediates for these compounds.

That is, the present invention relates to a compound of the formula I or its salt:

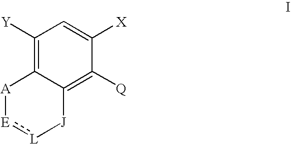

wherein X and Y are independent of each other and are hydrogen, halogen, cyano, nitro, (C₁₋₄)alkyl, (C₁₋₄)alkoxy, (C₁₋₄)haloalkyl or (C₁₋₄)haloalkoxy;

A is oxygen or CR₁R₂; E and L are independent of each other and are selected from CR₃, CR₃R₄, oxygen, nitrogen, NR₅, S(O)n, C(═O), C(═S), or C(═NR₆); J is oxygen or CR₇R₈; when A is CR₁R₂, J is oxygen; R₁, R₂, R₃, R₄, R₅, R₆, R₇ and R₈ are independent of each other and are selected from the group consisting of hydrogen, halogen, cyano, nitro, amino, hydroxy, mercapto, carboxyl, (C₁₋₁₂)alkyl, (C₁₋₆)alkoxy, (C₂₋₆)alkenyl, (C₂₋₆)alkynyl, (C₂₋₆)alkenyloxy, (C₂₋₆)alkynyloxy, aryl, heteroaryl, aryloxy, heteroaryloxy, (C₃₋₆)cycloalkyl, (C₃₋₆)cycloalkylcarbonyl, (C₁₋₆)alkylcarbonyl, arylcarbonyl, di(C₁₋₆alkyl)aminocarbonyl, (C₁₋₆)alkylcarbonyloxy, (C₁₋₆)alkoxyearbonyl, (C₁₋₆)alkylthiocarbonyl, (C₁₋₆)alkoxythiocarbonyl, (C₁₋₆)alkylthio, arylthio, (C₂₋₆)alkenylthio, (C₂₋₆)alkynylthio, (C₁₋₆)alkylsulfinyl, (C₂₋₆)alkenylsulfinyl, (C₂₋₆)alkynylsulfinyl, (C₁₋₆)alkylsulfonyl, (C₂₋₆)alkenylsulfonyl, (C₂₋₆)alkynylsulfonyl, arylsulfinyl, arylsulfonyl, —CR₁₃═NOR₁₄ and —CO₂NR₁₃R₁₄, where any of these groups may be substituted with one or more of the following group consisting of halogen, hydroxy, mercapto, cyano, nitro, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkylsulfonyloxy, (C₁₋₆)haloalkylsulfonyloxy, (C₁₋₆)alkoxy, (C₁₋₆)alkoxycarbonyl, aminocarbonyl, (C₁₋₆)alkylaminocarbonyl, di(C₁₋₆alkyl)aminocarbonyl, arylcarbonyl, haloarylcarbonyl, (C₁₋₆)haloalkoxy, (C₁₋₆)haloalkoxycarbonyl, (C₁₋₆)alkylsulfonyl, (C₁₋₆)haloalkylsulfonyl, arylsulfonyl, haloarylsulfonyl, aryl, haloaryl, alkoxyaryl, aryloxy, arylthio, haloaryloxy, heteroaryl, heteroaryloxy and (C₃₋₇)cycloalkyl; when R₃ and R₄ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, hydroxy, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, aryl, heteroaryl and (C₃₋₇)cycloalkyl; n is represent an integer from 0 to 2;

Q is selected from;

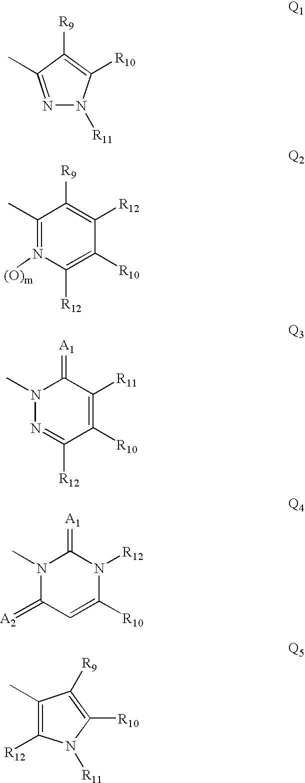

wherein A₁ and A₂ are independently oxygen or sulfur; R₉ and R₁₀ are independent of each other and are selected from the group consisting of halogen, cyano, nitro, formyl, hydroxy, amino, mercapto, (C₁₋₄)alkyl, (C₁₋₄)haloalkyl, (C₁₋₄)alkoxy, (C₁₋₄)haloalkoxy, (C₁₋₄)alkylamino, di(C₁₋₄alkyl)amino, (C₁₋₄)haloalkylamino, di(C₁₋₄haloalkyl)amino, (C₁₋₄)alkoxyamino, di(C₁₋₄alkoxy)amino, (C₁₋₄)haloalkoxyamino, di(C₁₋₄haloalkoxy)amino, (C₁₋₄)alkylcarbonyl, (C₁₋₄)haloalkylcarbonyl, (C₁₋₄)alkoxycarbonyl, (C₁₋₄)haloalkoxycarbonyl, (C₁₋₄)alkylcabonylamino, (C₁₋₄)haloalkylcarbonylamino, (C₁₋₄)alkoxycarbonylamino, (C₁₋₄)haloalkoxycarbonylamino, (C₁₋₆)alkoxyalkyl, (C₁₋₆)haloalkoxyalkyl, (C₁₋₆)alkoxyalkoxy, (C₁₋₆)alkylthio, (C₁₋₆)haloalkylthio, (C₂₋₆)alkenylthio, (C₂₋₆)haloalkenylthio, (C₂₋₆)alkynylthio, (C₂₋₆)haloalkynylthio, (C₁₋₆)alkylsulfinyl, (C₁₋₆)haloalkylsulfinyl, (C₂₋₆)alkenylsulfinyl, (C₂₋₆)haloalkenylsulfinyl, (C₂₋₆)alkynylsulfinyl, (C₂₋₆)haloalkynylsulfinyl, (C₁₋₆)alkylsulfonyl, (C₁₋₆)haloalkylsulfonyl, (C₂₋₆)alkenylsulfonyl, (C₂₋₆)haloalkenylsulfonyl, (C₂₋₆)alkynylsulfonyl, (C₂₋₆)haloalkynylsulfonyl, arylsulfonyl, (C₂₋₆)alkenyl, (C₂₋₆)haloalkenyl, (C₂₋₆)alkynyl and (C₂₋₆)haloalkynyl; R., is selected from the group consisting of hydrogen, halogen, (C₁₋₃)alkyl, (C₁₋₃)haloalkyl, hydroxy, (C₁₋₃)alkoxy, (C₁₋₃)haloalkoxy, cyano, nitro, amino, (C₁₋₃)alkylcarbonyl, (C₁₋₃)alkoxycarbonyl and (C₁₋₆)alkylamino; R₁₂ is selected from the group consisting of hydrogen, halogen, cyano, amino, (C₁₋₄)alkyl, (C₁₋₄)haloalkyl, (C₁₋₄)alkoxy, (C₁₋₄)haloalkoxy, (C₂₋₆)alkenyl, (C₂₋₆)haloalkenyl and hydroxy; when R₉ and R₁₀ or R₁₀ and R₁₁ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, hydroxy, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, aryl, heteroaryl and (C₃₋₇)cycloalkyl; m is 0 or 1; R₁₃ and R₁₄ are independent of each other and are selected from the group consisting of hydrogen, (C₁₋₆)alkyl and aryl; provided that A is oxygen and J is CR₇R₈, when Q is Q₄.

Preferred compounds for the reasons of greater herbicidal efficacy are represented by formula I where;

(1) X and Y are independent of each other and are hydrogen, halogen or cyano;

A is oxygen; E and L are independent of each other and are selected from CR₃, CR₃R₄, oxygen, nitrogen, NR₅, S(O)n or C(═O); J is CR₇R₈; R₃, R₄, R₅, R₇ and R₈ are independent of each other and are selected from the group consisting of hydrogen, halogen, cyano, nitro, amino, hydroxy, (C₁₋₆)alkyl, (C₁₋₆)alkoxy, (C₂₋₆)alkenyl, (C₂₋₆)alkynyl, aryl, aryloxy, (C₃₋₆)cycloalkyl, (C₁₋₆)alkylcarbonyl, arylcarbonyl, (C₁₋₆)alkylcarbonyloxy, (C₁₋₆)alkoxycarbonyl, (C₁₋₆)alkylthiocarbonyl, (C₁₋₆)alkoxythiocarbonyl, (C₁₋₆)alkylthio, arylthio, (C₂₋₆)alkenylthio, (C₂₋₆)alkynylthio, (C₁₋₆)alkylsulfinyl, (C₂₋₆)alkenylsulfinyl, (C₂₋₆)alkynylsulfinyl, (C₁₋₆)alkylsulfonyl, (C₂₋₆)alkenylsulfonyl, (C₂₋₆)alkynylsulfonyl, arylsulfinyl and arylsulfonyl, where any of these groups may be substituted with one or more of the following group consisting of halogen, hydroxy, mercapto, cyano, nitro, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, arylsulfonyl, aryl, haloaryl, alkoxyaryl, aryloxy, arylthio, haloaryloxy, heteroaryl, heteroaryloxy and (C₃₋₇)cycloalkyl; n is represent an integer from 0 to 2;

Q is Q₁, Q₂ or Q₅; R₉ and R₁₀ are independent of each other and are selected from the group consisting of halogen, cyano, formyl, hydroxy, amino, mercapto, (C₁₋₄)alkyl, (C₁₋₄)haloalkyl, (C₁₋₄)alkoxy, (C₁₋₄)haloalkoxy, (C₁₋₄)alkylamino, (C₁₋₄)haloalkylamino, (C₁₋₄)alkoxyamino, (C₁₋₄)haloalkoxyamino, (C₁₋₄)alkylcarbonyl, (C₁₋₄)haloalkylcarbonyl, (C₁₋₄)alkoxycarbonyl, (C₁₋₄)haloalkoxycarbonyl, (C₁₋₄)alkylcabonylamino, (C₁₋₄)haloalkylcarbonylamino, (C₁₋₄)alkoxycarbonylamino, (C₁₋₄)haloalkoxycarbonylamino, (C₁₋₆)alkoxyalkyl, (C₁₋₆)haloalkoxyalkyl, (C₁₋₆)alkylthio, (C₁₋₆)haloalkylthio, (C₂₋₆)alkenylthio, (C₂₋₆)haloalkenylthio, (C₂₋₆)alkynylthio, (C₂₋₆)haloalkynylthio, (C₁₋₆)alkylsulfinyl, (C₁₋₆)haloalkylsulfinyl, (C₂₋₆)alkenylsulfinyl, (C₂₋₆)haloalkenylsulfinyl, (C₂₋₆)alkynylsulfinyl, (C₂₋₆)haloalkynylsulfinyl, (C₁₋₆)alkylsulfonyl, (C₁₋₆)haloalkylsulfonyl, (C₂₋₆)alkenylsulfonyl, (C₂₋₆)haloalkenylsulfonyl, (C₂₋₆)alkynylsulfonyl, (C₂₋₆)haloalkynylsulfonyl, arylsulfonyl, (C₂₋₆)alkenyl, (C₂₋₆)haloalkenyl, (C₂₋₆)alkynyl and (C₂₋₆)haloalkynyl; R is selected from the group consisting of hydrogen, (C₁₋₃)alkyl, (C₁₋₃)haloalkyl, (C₁₋₃)alkylcarbonyl and (C₁₋₃)alkoxycarbonyl; R₁₂ is selected from the group consisting of hydrogen, halogen, cyano, (C₁₋₄)alkyl, (C₁₋₄)haloalkyl, (C₁₋₄)alkoxy, (C₁₋₄)haloalkoxy, (C₂₋₆)alkenyl, (C₂₋₆)haloalkenyl and hydroxy; when R₉ and R₁₀ or R₁₀ and R₁₁ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, hydroxy, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, aryl, heteroaryl and (C₃₋₇)cycloalkyl.

(2) X and Y are independent of each other and are hydrogen, halogen or cyano;

A is oxygen; E and L, respectively, are CR₃R₄ and CR₃′R₄′; J is CR₇R₈; R₃, R₄, R₃′, R₄′, R₇ and R₈ are independent of each other and are selected from the group consisting of hydrogen, halogen, cyano, nitro, hydroxy, (C₁₋₆)alkyl, (C₁₋₆)haloalkyl, (C₁₋₆)alkoxy, (C₁₋₆)haloalkoxy, (C₁₋₆)alkoxyalkyl, (C₂₋₆)alkenyl, (C₂₋₆)alkynyl, aryl, aryloxy, (C₃₋₆)cycloalkyl, (C₁₋₆)alkylcarbonyl, arylcarbonyl, (C₁₋₃)haloalkylcarbonyl, (C₁₋₆)alkylcarbonyloxy, (C₁₋₆)haloalkylcarbonyloxy, (C₁₋₆)alkoxycarbonyl, (C₁₋₆)haloalkoxycarbonyl, (C₁₋₆)alkylthiocarbonyl, (C₁₋₆)haloalkylthiocarbonyl, (C₁₋₆)alkoxythiocarbonyl, (C₁₋₆)haloalkoxythiocarbonyl, arylsulfonylamino, arylamino, (C₁₋₆)alkylthio, arylthio, (C₂₋₆)alkenylthio, (C₂₋₆)alkynylthio, (C₁₋₆)alkylsulfinyl, (C₂₋₆)alkenylsulfinyl, (C₂₋₆)alkynylsulfinyl, (C₁₋₆)alkylsulfonyl, (C₂₋₆)alkenylsulfonyl, (C₂₋₆)alkynylsulfonyl, arylsulfinyl and arylsulfonyl;

Q is Q₁ or Q₂; R₉ and R₁₀ are independent of each other and are selected from the group consisting of halogen, cyano, hydroxy, amino, (C₁₋₄)alkyl, (C₁₋₄)haloalkyl, (C₁₋₄)alkoxy and (C₁₋₄)haloalkoxy; R₁, is selected from the group consisting of hydrogen, (C₁₋₃)alkyl and (C₁₋₃)haloalkyl; R₁₂ is selected from the group consisting of hydrogen, halogen, cyano, (C₁₋₄)alkyl, (C₁₋₄)haloalkyl, (C₁₋₄)alkoxy and (C₁₋₄)haloalkoxy; when R₉ and R₁₀ or R₁₀ and R₁₁ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, (C₁₋₆)alkyl, (C₁₋₆)haloalkyl and (C₁₋₆)alkoxy; m is 0; n is represent an integer from 0 to 2.

(3) X and Y are independent of each other and are hydrogen or halogen;

A is oxygen; F and L, respectively, are CR₃R₄ and CR₃′R₄′; J is CH₂; R₃, R₄, R₃′, and R₄′ are independent of each other and are selected from the group consisting of hydrogen, halogen, cyano, (C₁₋₆)alkyl, (C₁₋₆)haloalkyl, (C₁₋₆)alkoxy, (C₁₋₆)haloalkoxy, (C₁₋₆)alkoxyalkyl, (C₂₋₆)alkenyl, aryl, aryloxy, (C₃₋₆)cycloalkyl, (C₁₋₆)alkylcarbonyl, (C₁₋₃)haloalkylcarbonyl, (C₁₋₆)alkylcarbonyloxy, (C₁₋₆)haloalkylcarbonyloxy, (C₁₋₆)alkoxycarbonyl, (C₁₋₆)alkylthiocarbonyl, (C₁₋₆)alkoxythiocarbonyl, (C₁₋₆)haloalkoxythiocarbonyl, (C₁₋₆)alkylthio, arylthio, (C₂₋₆)alkynylthio, (C₁₋₆)alkylsulfinyl and (C₁₋₆)alkylsulfonyl;

Q is Q, or Q₂; R₉ and R₁₀ are independent of each other and are selected from the group consisting of halogen, (C₁₋₄)alkyl, (C₁₋₄)haloalkyl and (C₁₋₄)haloalkoxy; R., is (C₁₋₃)alkyl; R₁₂ is selected from the group consisting of hydrogen and halogen; when R₉ and R₁₀ or R₁₀ and R₁₁ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, (C₁₋₆)alkyl, (C₁₋₆)haloalkyl and (C₁₋₆)alkoxy; m is 0; and n is represent an integer from 0 to 2.

In the definitions given above, unless alkyl, alkenyl, alkynyl and halogen are defined or mentioned, the term alkyl used either alone or in compound words such as “haloalkyl”, “haloalkoxy”, “alkoxythio” or “alkylcarbonyl” includes straight-chain or branched chains. The terms of alkenyl and alkynyl include straight chain or branched alkenes and alkynes respectively containing 2 to 6 carbon atoms, and the term halogen either alone or in the compound words such as haloalkyl indicates fluorine, chlorine, bromine, or iodine.

Further a haloalkyl is represented by an alkyl partially or fully substituted with halogen atoms which may be same or different. The term or part of the term “aryl” or “heteroaryl” are defined as those monocyclic or fused bicyclic aromatic rings wherein at least one ring satisfy the Hückel rule and contain 0 to 4 heteroatoms. The aryl and heteroaryl rings are preferably 5 to 10-membered rings, and the heteroaryl ring has at least one heteroatoms of nitrogen, oxygen or sulfur. Examples include: phenyl, furyl, furazanyl, thienyl, pyrrolyl, pyrazolyl, oxazolyl, oxadiazolyl, imidazolyl, isoxazolyl, thiazolyl, thiadiazolyl, isothiazolyl, tetrazolyl, pyridinyl, pyrimidinyl, pyridazinyl, pyrazinyl, triazinyl, quinolyl, isoquinolyl, quinoxalinyl, benzofuranyl, 2,3-dihydrobenzofuranyl, isobenzofuranyl, benzothienyl, benzodioxolyl, chromanyl, indolinyl, isoindolyl, naphthyl, thienofuranyl and purinyl. These rings can attached through any available carbon or nitrogen, for example, when the aromatic ring system is furyl, it can be 2-furyl or 3-furyl, for pyrrolyl, the aromatic ring system is 1-pyrrolyl, 2-pyrrolyl or 3-pyrrolyl, for naphthyl, the carbobicyclic aromatic ring is 1-naphthyl or 2-naphthyl and for benzofuranyl, the aromatic ring system can be 2-, 3-, 4-, 5-, 6- or 7-benzofuranyl.

### DETAILED DESCRIPTION OF THE INVENTION

The compounds described by the Formula I can be prepared by the procedures as described herein. Using commercially available starting materials or those whose synthesis is known in the art, the compounds of this invention may be prepared using methods described in the following Schemes, or using modifications thereof, which are within the scope of the art.

The starting phenol derivatives represented by formula II in Scheme 1 can be prepared according to the literature procedure (e.g. EP 361114). The propargyl ether derivatives represented by formula III can be prepared by known method. The reaction can be carried out with or without catalyst such as sodium iodide in the presence inorganic or organic base such as potassium carbonate, sodium hydroxide, triethylamine or 1,8-diazabicyclo[5.4.0]undec-7-ene (DBU) in an inert solvent such as toluene, 2-butanone, N,N-dimethylformamide (DMF) or dimethylsulfoxide (DMSO) at a temperature between 0° C. and 200° C. for 0.5 to 24 hours. The ethers represented by formula III can also be prepared by the condensation reaction of II with the corresponding butyn-2-ol by using dehydrating reagent such as 1,3-dicyclohexylcarbodiimide (DCC) or ethyl azodicarboxylate in the presence of base such as N,N-dimethylaminopyridine (DMAP) and/or phosphine such as triphenyl phosphine. The reaction can be carried out in an inert solvent such as dichloromethane or tetrahydrofurane (THF) at a temperature between −5° C. and 100° C. for 0.5 to 48 hours.

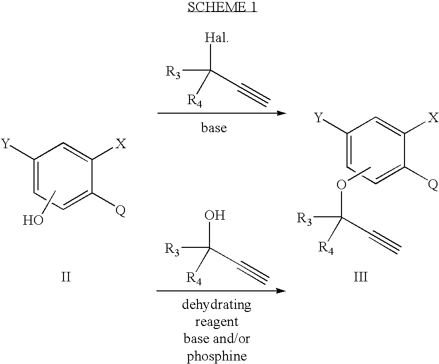

The compounds represented by formula III in Scheme 2 are prepared from the corresponding alcohol according to the literature procedure (J. D. Golfrey et al., Tetrahedron Lett. 1994, 35, 6405-6408). The reaction can be carried out in the presence of catalyst such as copper (II) chloride with an acid chloride such as acetyl chloride or acid anhydride such as trifluoroacetic anhydride in an inert solvent such as acetonitrile at a temperature between −10° C. and 100° C. for 0.5 to 48 hours. The reaction can be accelerated by a base such as DBU. The solvent is removed under reduced pressure, then isolated and purified.

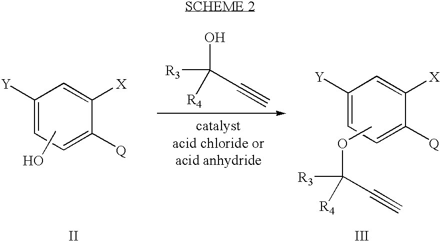

The compounds represented by formula V in Scheme 3 can be prepared by the cyclization reaction of IV. The reaction can be carried out in an inert solvent such as N,N-dietylaniline or m-xylene at a temperature between 50° C. and 250° C. for 0.5 to 24 hours with or without additive such as cesium carbonate. The chroman derivatives represented by formula VI can be prepared from V by catalytic hydrogenation. The reaction can be carried out in the presence of catalyst such as palladium-carbon or platinumoxide under hydrogen atmosphere. The reaction can be carried out in an inert solvent such as ethyl acetate at a temperature between 0° C. and 150° C. for 0.5 to 48 hours. The solvent is removed and then isolated and purified to give VI.

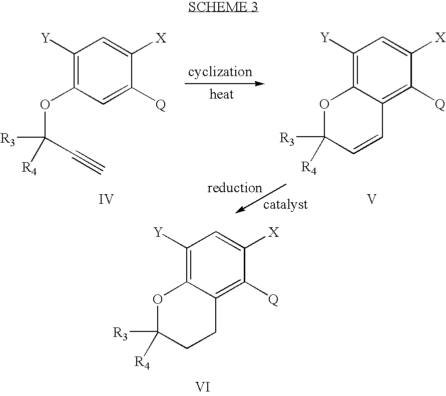

The diole derivatives represented by formula VII and IX in Scheme 4 can be prepared according to the literature procedure (e.g. U.S. Pat. No. 4,881,967). The chroman derivatives represented by formula VIII and X can be prepared from diole derivatives represented by formula VII and IX respectively in Scheme 4. The reaction can be carried out in the presence of acid such as p-toluenesulfonic acid in an inert solvent such as toluene at a temperature between 0° C. and 200° C. for 0.5 to 48 hours. Alternatively, VII and IX can be treated by dehydrating reagent such as diethyl azodicarboxylate in the presence of phosphine such as triphenylphosphine. The reaction can be carried out in an inert solvent such as THF at a temperature between −10° C. and 100° C. for 0.5 to 48 hours.

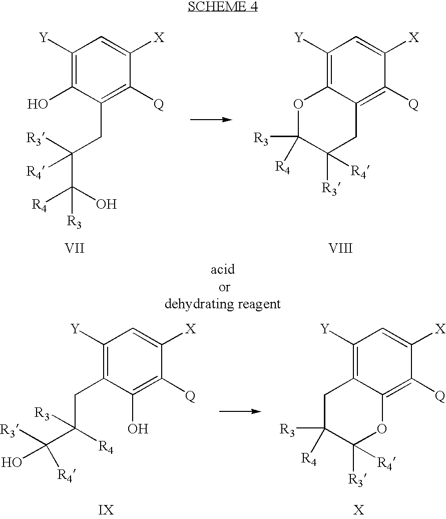

The chroman derivatives represented by formula VI in Scheme 5 can be prepared from phenol derivatives represented by formula II′ according to the literature procedure (V. K. Ahluwalia et al., Indian Journal of Chemistry, 1984, 23B 129-131). II′ can be reacted with an olefinic compound such as isoprene, or an alcohol derivative such as 2-methylbut-3-en-2-ol in the presence of acid such as phosphoric acid. The reaction can be carried out in an inert solvent such as petroleum ether at a temperature between 25° C. and 200° C. for 0.5 to 48 hours. The solvent was removed under reduced pressure, and then isolated and purified.

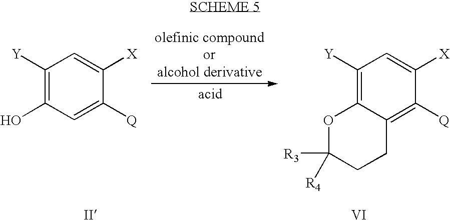

The chroman derivatives represented by formula XIV in Scheme 6 can be prepared from XI through the corresponding chromene derivatives represented by formula XIII according to the general procedure as described in Scheme 1 to 3. XIV is then converted into the corresponding nitro derivatives represented by formula XV. Nitration can be carried out by treatment with a nitrating reagent such as nitric acid at a temperature between −40° C. and 100° C. for 0.5 to 48 hours. The aniline derivatives represented by formula XVII can be prepared from XV by typical reduction procedure e.g. iron in an acidic medium such as acetic acid or by catalytic hydrogenation.

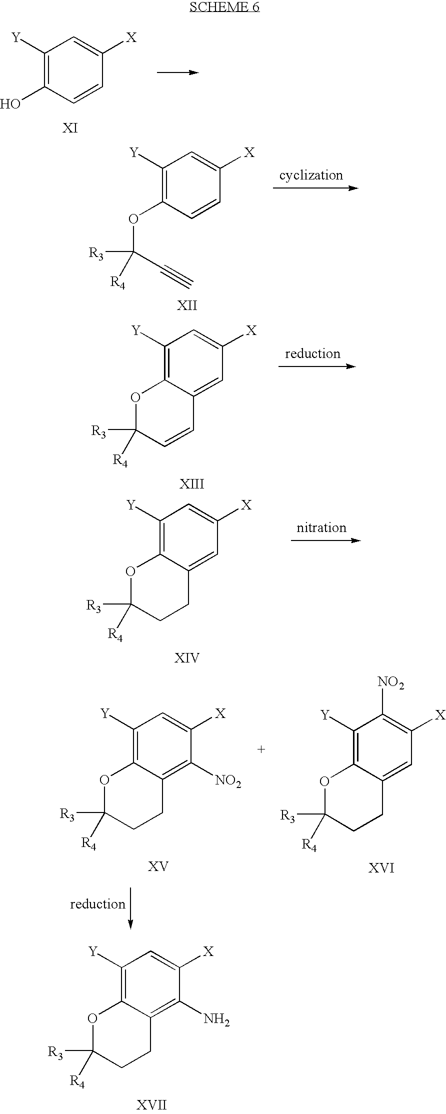

The chroman derivatives represented by formula XXI in Scheme 7 can be prepared from XVIII according to the general procedure described in Scheme 1 to 3. XXI is then converted into the corresponding acid derivatives represented by formula XXII by oxidation procedure. The oxidation reaction can be carried out in the presence metal salt catalyst or oxidant such as cobalt salts, nickel salts, manganese salts, zirconium salts, chromium salts, cesium salts or vanadium salts individually or in combination. Any known reaction initiator such as hydrogen peroxide, benzoyl peroxide or oxygen is suitable to initiate the oxidation reaction. A catalyst promoter such as sodium bromide, calcium acetate, hydrogen bromide or ammonium salts such as tetrabutyl ammonium bromide can be used in conjunction with metal salt catalyst. The reaction can be carried out with or without solvent such as water, acetic acid, acetone or halo-carbon such as dichloroethane at a temperature between 0° C. and 250° C. for 0.5 to 72 hours. The product can be isolated and purified. The acid derivatives represented by formula XXII can be converted into XXVI according to the literature procedure (JP 04225937).

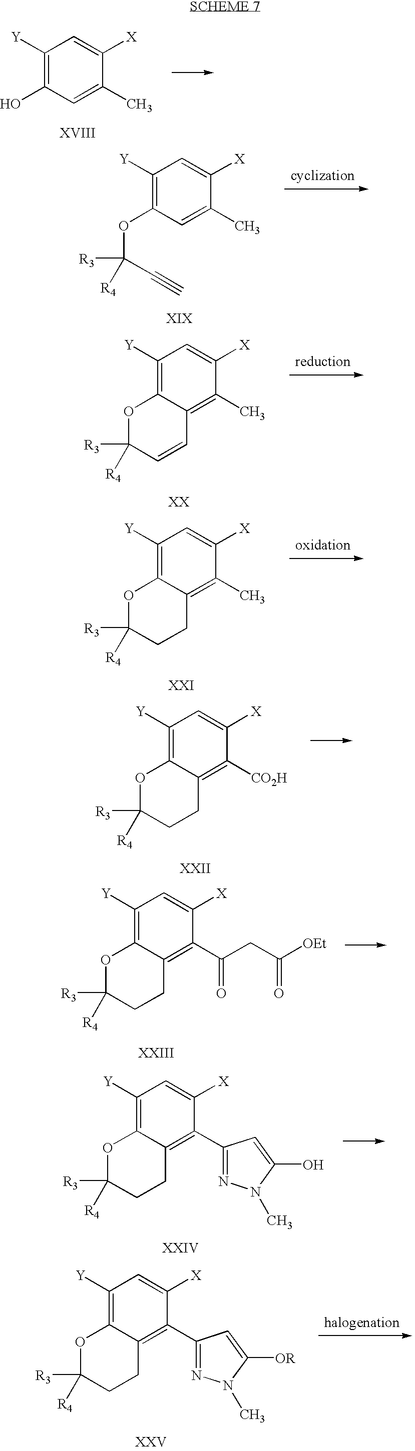

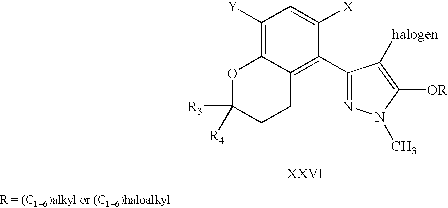

The uracil derivatives represented by formula XXIX in Scheme 8 can be prepared analogously by known methods (EP 255047, JP 10218862). The anilines described by formula XVII is treated with phosgene or triphosgene in the presence of base such as triethyl amine. The reaction can be carried out in an inert solvent such as ethyl acetate or toluene at a temperature between −10° C. and 150° C. for 0.5 to 24 hours. XXVII can be treated with crotonate in the presence of base such as sodium hydride, sodium ethoxide, potassium butoxide, sodium hydroxide, potassium hydroxide, triethyl amine or DBU in an inert solvent such as toluene, xylene or N,N-dimethylformamide at a temperature between −10° C. and 200° C. for 0.5 to 48 hours. The compounds represented by formula XXVIII can be an alternative intermediate. XVII is converted into XXVIII by the treatment with chloroformate such as ethyl chloroformate in the presence of base such as triethylamine. XXIX is synthesized analogously according to the synthesis method described above.

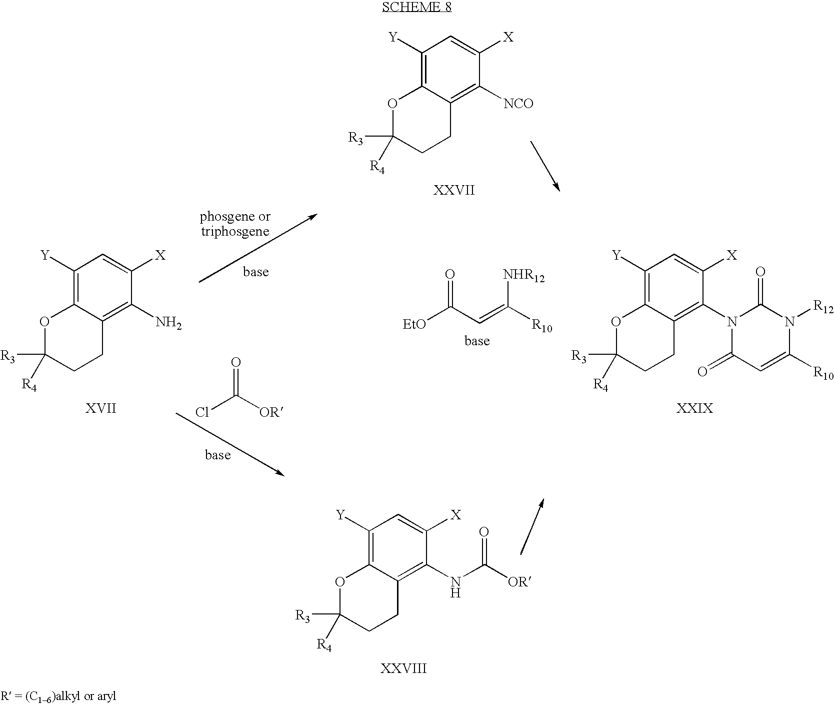

The chroman derivatives represented by formula XXXII in Scheme 9 can be prepared from XXXI according to the general procedure described in Scheme 3. The organometallic compounds represented by formula XXXIII can be prepared analogously by known methods (e.g. WO 01/10843). The pyridine derivatives represented by formula XXXIV can be prepared analogously by known method (WO 98/11072, U.S. Pat. No. 6,169,184). The reaction can be carried out with or without transition metal catalyst such as dichlorobis(triphenylphosphine)palladium, tetrakis(triphenylphosphine)palladium or dichlorobis(triphenylphosphine)nickel in an inert solvent such as THF, benzene, toluene, diethyl ether, hexane, xylene, N,N-dimethylformamide or dimethylsulfoxide at a temperature between −10° C. and 150° C. for 0.5 to 48 hours.

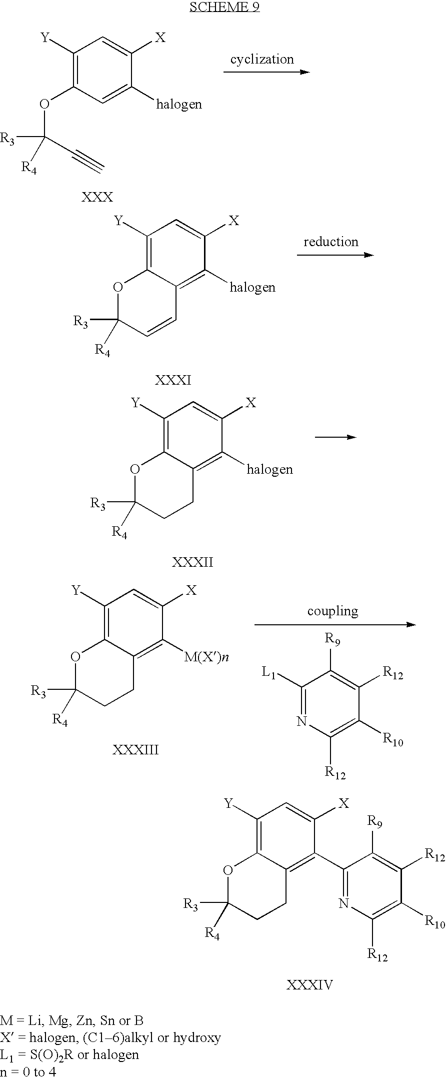

The compounds represented by formula XXXVI in Scheme 10 can be prepared according to the general procedure described in Scheme 3. The reduction of XXXVI to aniline derivatives represented by formula XVII can be carried out by catalytic hydrogenation at a temperature between 0° C. and 100° C. for 0.5 to 48 hours. Another intermediates represented by formula XXXVIII can be prepared through XXXVII analogously as described in Scheme 3. XVII can be prepared from XXXVIII by catalytic hydrogenation at a temperature between 0° C. and 100° C. for 0.5 to 48 hours.

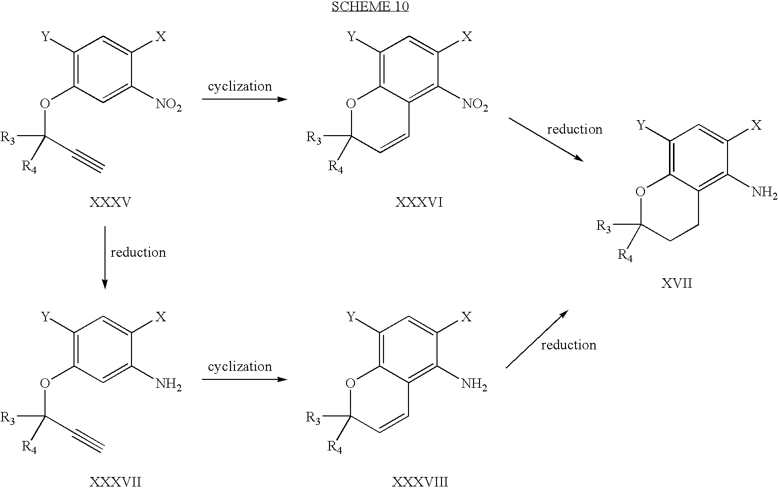

The hydrazine derivatives represented by formula XXXIX in Scheme 11 can be prepared from XVII according to the literature procedure (e.g. Organic Synthesis Collective, Vol. 1, p.442). XXXXI can be prepared from XXXIX through XXXX analogously according to the literature procedure (WO 97/07104).

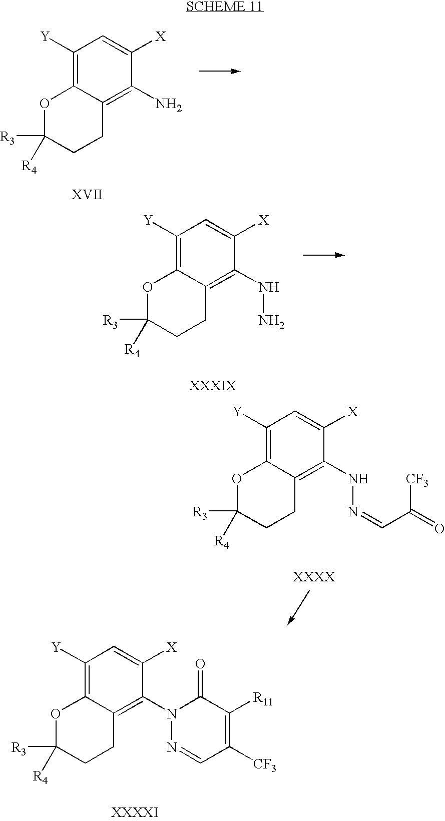

The starting compounds represented by formula XXXXII in Scheme 12 can be prepared analogously according to the literature procedure (WO 93/15074). The pyrrol derivatives represented by formula XXXXIII can be prepared analogously by known method (EP 1061072). The condensation reaction with the corresponding amino-acid derivatives represented by formula XXXXV can be carried out in the presence of dehydrating reagent such as acetic anhydride in an inert solvent such as hexane, toluene, xylene at a temperature between 25° C. and 250° C. for 0.5 to 48 hours. The final compounds represented by formula XXXXIV can be prepared from XXXXIII by treatment with a halogenation reagent such as thionyl chloride, bromine, N-chlorosuccinimide or N-bromosuccinimide in an inert solvent such as 1,4-dioxane or N,N-dimethylformamide at a temperature between 25° C. and 200° C. for 0.5 to 24 hours.

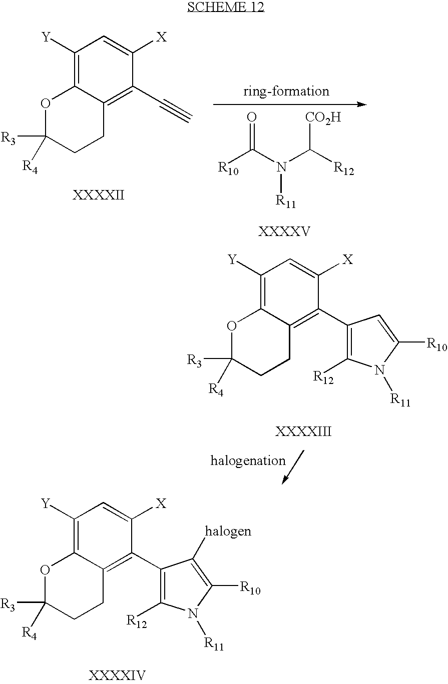

The acid derivatives represented by formula XXII in Scheme 13 can be prepared from XXXII through XXXIII by using transmetallation procedure. XXXII can be treated with a organometallic compound such as n-butyllithium or isobutylmagnesium chloride in an inert solvent such as THF or diethylether, with or without co-solvent such as hexamethylphosphoramide (HMPA) at a temperature between −78° C. and 25° C. for 10 to 120 minutes. After complete formation of XXXIII, an electrophile such as dry-ice or CO₂ (gas) can be added to the reaction solution at a temperature between −78° C. and 80° C. to form XXII. The product was isolated and purified.

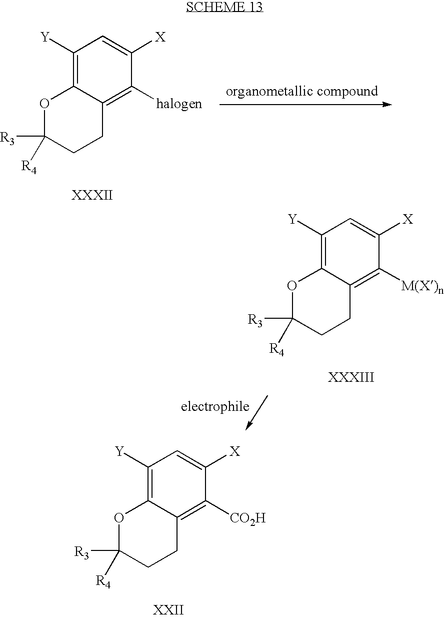

The chroman derivatives represented by formula XXXXVII in Scheme 14 can be prepared from XIV. The acetylation reaction of XIV can be carried out with an acid chloride such as acetylchloride in the presence of Lewis acid such as aluminum chloride in an inert solvent such as dichlomethane at a temperature between −10° C. and 150° C. for 0.5 to 48 hours.

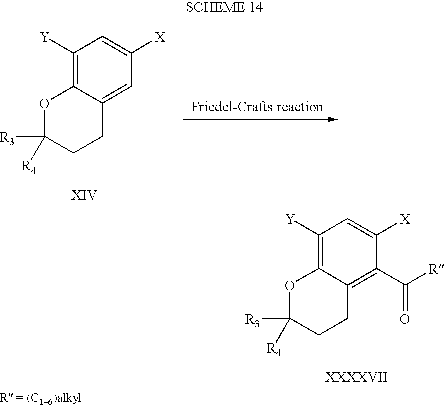

The compounds represented by formula XXXXVI in Scheme 15 can be prepared analogously according to the general procedure described in Scheme 14. The reduction of XXXXVI to chroman derivatives represented by formula XXXXVII can be carried out by catalytic hydrogenation at a temperature between 0° C. and 100° C. for 0.5 to 48 hours.

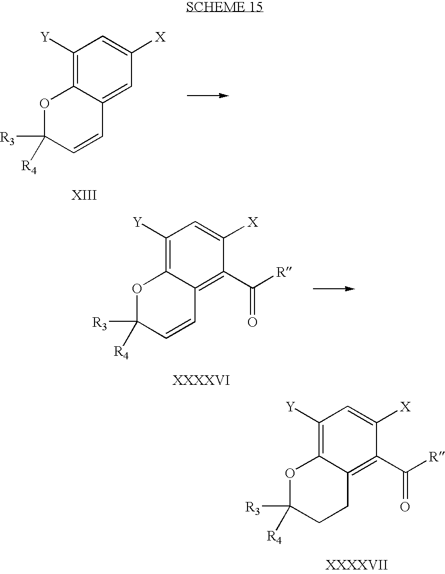

XXXXVIII in Scheme 16 can be prepared according to the general procedure described in Scheme 14 to 15. XXXXIX can be prepared analogously by known method (WO 92/06962). XXXXVIII may be treated with an ester such as ethyl trifluoroacetate in the presence of base such as sodium hydride in an inert solvent such as N,N-dimethylformamide or THF. The reaction can be carried out at a temperature between −30° C. and 150° C. for 0.5 to 24 hours. Pyrazole derivatives represented by formula L can be prepared from XXXXIX by treatment with a hydrazine derivative such as hydrazine dihydrate in an inert solvent such as ethanol. Reaction temperature is in the range of −40° C. to 150° C. for 0.5 to 24 hours. The resulting pyrazole compounds of formula L may be treated with an alkylating reagent such as methyl iodide or dimethylsulfoxide to obtain compound represented by formula LI. The reaction can be carried out with or without base such as potassium carbonate in an inert solvent such as toluene, N,N-dimethylformamide, acetone or dioxane at a temperature between 0° C. and 200° C. for 0.5 to 24 hours. LII can be prepared from LI by the halogenation procedure. The reaction can be carried out in the presence of halogenating reagent such as chlorine, bromine, N-chlorosuccinimide or sulfuryl chloride in an inert solvent such as acetic acid, N,N-dimethylformamide, dimethylsulfoxide, toluene or THF. The reaction temperature is in the range between 0° C. and 200° C. for 0.5 to 24 hours.

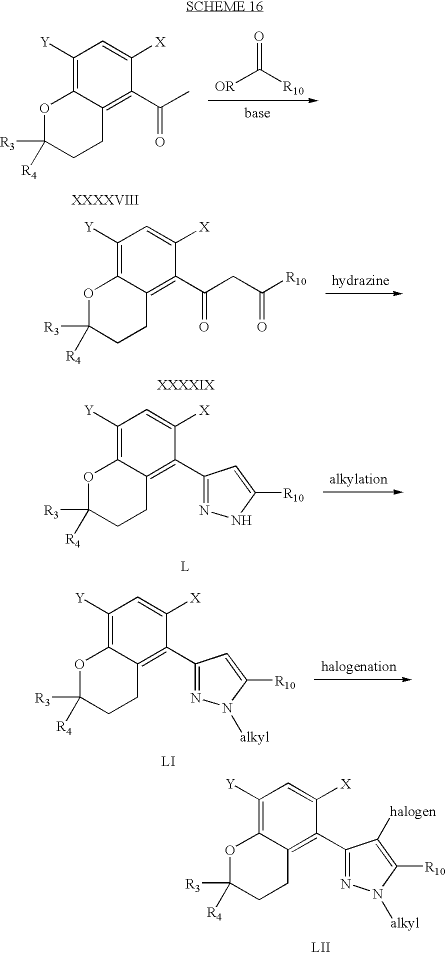

The aniline derivatives represented by formula LV in Scheme 17 can be prepared from LIII through LIV according to the literature procedure (WO98/38188). The propionic acid derivative represented by formula LVI can be prepared analogously by known method (WO99/21837). The benzopyrane derivatives represented by formula LVII can be prepared from LVI by treatment with a catalyst such as p-toluenefulfonic acid or triethylamine in an inert solvent such as toluene or THF. The reaction can be carried out at a temperature between −10° C. and 150° C. for 0.5 to 24 hours. The dihydro-benzopyran derivatives represented by formula LVIII can be prepared from LVII under reduction condition such as catalytic hydrogenation.

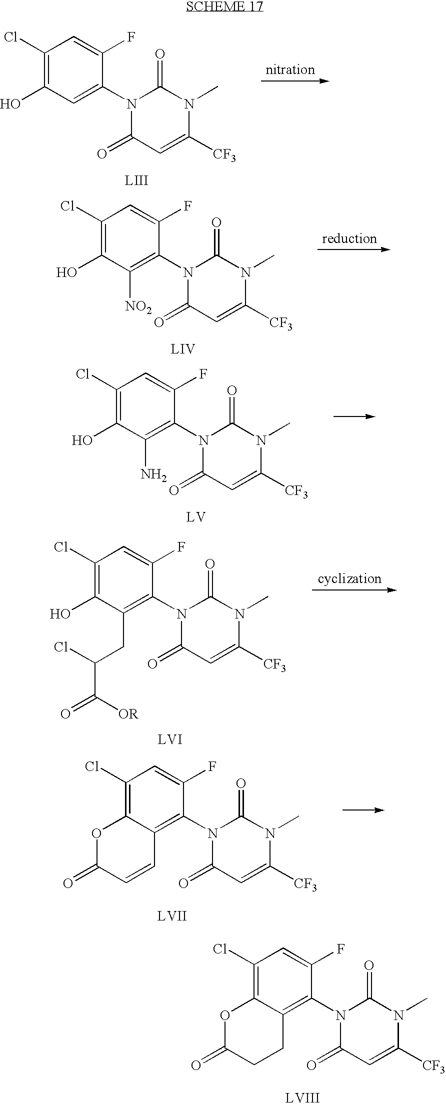

### EXAMPLE 1

Preparation of 3-chloro-2-(8-chloro-6-fluoro-3,4-dihydro-2H-1-benzopyran-5-yl)-4,5,6,7-tetrahydro-pyrazolo[1,5-a]pyridine (Compound No. 1-79)

Step 1 Preparation of 3-chloro-2-[4-chloro-2-fluoro-5-(2-propynyloxy)phenyl]-4,5,6,7-tetrahydro-pyrazolo[1,5-a]pyridine as an Intermediate

Potassium carbonate (0.65 g) was added a solution of 2-chloro-5-(3-chloro-4,5,6,7-tetrahydropyrazolo[1,5-a]pyridin-2-yl)-4-fluorophenol (1 g) and propargyl bromide (1 g) in a mixed solvent of 2-butanone and dimethylsulfoxide (3:1, 30 ml) at ambient temperature. The mixture was heated at refluxed temperature for 30 minutes and then allowed to cool to room temperature. The solution was partitioned between ethyl acetate and brine. The organic layer was dried over anhydrous sodium sulfate and concentrated in vacuo to afford the titled compound as a brown solid.

¹H-NMR (CDCl₃, 400 MHz): 1.90 (2H, m), 2.05 (2H, m), 2.52 (1H, br s), 2.75 (2H, t, J=6.4 Hz), 4.13 (2H, t, J=5.2 Hz), 4.75 (2H, s), 7.21 (1H, d, J=9.2 Hz), 7.26 (1H, d, J=6.4 Hz).

Step 2 Preparation of 3-chloro-2-(8-chloro-6-fluoro-2H-1-benzopyran-5-yl)-4,5,6,7-tetrahydro-pyrazolo[1,5-a]pyridine as an Intermediate

3-Chloro-2-[4-chloro-2-fluoro-5-(2-propynyloxy)phenyl]-4,5,6,7-tetrahydro-pyrazolo[1,5-a]pyridine (1 g) was dissolved in N,N-diethylaniline (8 ml). The solution was heated at 210° C. for 2 hours under nitrogen atmosphere. The mixture was diluted with ethyl acetate (200 ml) and washed with 2N-HCl (200 ml×2). The organic phase was dried over anhydrous sodium sulfate and concentrated in vacuo. The crude oily material was purified by column chromatography on silica gel eluted with a mixed solvent of ethyl acetate and hexane (1:3) to give the titled compound (0.65 g) as a colorless oil.

¹H-NMR (CDCl₃, 400 MHz): 1.9 (2H, m), 2.0 (2H, m), 2.70 (2H, t, J=6.1 Hz), 4.10 (2H, t, J=6 Hz), 4.81 (2H, dd, J=4.0, 2.0 Hz), 5.79 (1H, m), 6.30 (1H, dt, J=9.6, 1.8 Hz), 6.95 (1H, d, J=9.2 Hz).

Step 3 Palladium on carbon (5%, 0.1 g) was added to a solution of 3-chloro-2-(8-chloro-6-fluoro-2H-1-benzopyran-5-yl)-4,5,6,7-tetrahydro-pyrazolo[1,5-a]pyridine (0.3 g) in ethyl acetate (30 ml). The suspension was vigorously stirred under hydrogen atmosphere for 3 hours at ambient temperature. After filtration and evaporation, 3-chloro-2-(8-chloro-6-fluoro-3,4-dihydro-2H-1-benzopyran-5-yl)-4,5,6,7-tetrahydro-pyrazolo[1,5-a]pyridine (292 mg) was obtained as a single product.

Operating analogously to the procedure described in EXAMPLE 1, the following compounds were prepared;

3-chloro-2-(8-chloro-6-fluoro-3,4-dihydro-2H-1-benzopyran-5-yl)-5-(trifluoromethyl)-pyridine (Compound No. 7-2)

4-chloro-3-(8-chloro-6-fluoro-3,4-dihydro-2H-1-benzopyran-5-yl)-1-methyl-5-(trifluoromethyl)-1H-pyrazole (Compound No. 1-2)

4-chloro-3-(8-chloro-6-fluoro-3,4-dihydro-2H-1-benzopyran-5-yl)-5-(difluoromethoxy)-1-methyl-1H-pyrazole (Compound No. 1-33)

### EXAMPLE 2

Preparation of 4-chloro-3-(8-chloro-6-fluoro-3,4-dihydro-2,2-dimethyl-2H-1-benzopyran-5-yl)-5-(difluoromethoxy)-1-methyl-1H-pyrazole (Compound No. 1-40)

Step 1 Preparation of 4-chloro-3-[4-chloro-5-[(1,1-dimethyl-2-propynyl)oxy]-2-fluorophenyl]-5-(difluoromethoxy)-1-methyl-1H-pyrazole as an Intermediate

To a solution of trifluoroacetic anhydride (0.26 g) and 2-methyl-3-butyn-2-ol (0.154 mg) in acetonitrile (30 ml) was added DBU (0.58 g) at 0° C. After 10 minutes, 2-chloro-5-(4-chloro-5-difluoromethoxy-1-methyl-1H-pyrazol-3-yl)-4-fluoro-phenol (0.5 g) was added. The mixture was stirred for 2 hours at same temperature and then catalytic amount of copper (II) chloride dihydrate was added. The reaction mixture was stirred for 5 hours at ambient temperature. Ethyl acetate (200 ml) was added and washed with sat. brine (×2). The organic phase was dried over anhydrous sodium sulfate and concentrated in vacuo. The residue was subjected to column chromatography on silica gel eluted with a mixed solvent of ethyl acetate and hexane (15:85) to afford the titled compound (200 mg) as a colorless oil.

¹H-NMR (CDCl₃, 400 MHz): 1.69 (614, s), 2.58 (1H, s), 3.83 (3H, s), 6.70 (1H, t, J=72.2 Hz), 7.21 (1H, d, J=9.2 Hz), 7.77 (1H, d, J=6.8 Hz).

Step 2 Preparation of 4-chloro-3-(8-chloro-6-fluoro-2,2-dimethyl-2H-1-benzopyran-5-yl)-5-(difluoromethoxy)-1-methyl-1H-pyrazole as an Intermediate

A solution of 4-chloro-3-[4-chloro-5-[(1,1-dimethyl-2-propynyl)oxy]-2-fluorophenyl]-5-(difluoromethoxy)-1-methyl-1H-pyrazole (0.2 g) in N,N-diethylamine (10 ml) was heated at 210° C. for 2 hours under nitrogen atmosphere. The solution was allowed to cool to room temperature and diluted with ethyl acetate (200 ml). The organic phase was washed with 2N-HCl (200 ml×2) and dried over anhydrous sodium sulfate. The solvent was removed in vacuo to give the titled compound.

¹H-NMR (CDCl₃, 400 MHz): 1.41 (6H, s), 3.76 (3H, s), 5.64 (1H, d, J=9.6 Hz), 6.15 (1H, d, J=9.6 Hz), 6.45 (1H, t, J=72.4 Hz), 6.96 (1H, d, J=8.8 Hz).

Step 3 4-Chloro-3-(8-chloro-6-fluoro-2,2-dimethyl-2H-1-benzopyran-5-yl)-5-(difluoromethoxy)-1-methyl-1H-pyrazole (0.2 g, crude) was dissolved in ethyl acetate (30 ml) and palladium on carbon (5%, 0.1 g) was added. The resulting mixture was vigorously stirring under hydrogen atmosphere for 3 hours at ambient temperature. The reaction mixture was filtered through Celite and the filtrate was concentrated in vacuo to give the titled compound (165 mg) as a solid.

### EXAMPLE 3

Preparation of 3-(8-chloro-6-fluoro-3,4-dihydro-2H-1-benzopyran-5-yl)-1-methyl-6-(trifluoromethyl)-2,4 (1H,3H)-pyrimidinedione (Compound No.3-2)

Step 1 Preparation of 8-chloro-6-fluoro-2H—1-benzopyran-5-amine as an Intermediate

A solution of 4-chloro-2-fluoro-5-(2-propynyloxy)-benzenamine (0.89 g) in N,N,-diethylaniline (13 ml) was heated at 210° C. for two hours under nitrogen atmosphere. The resulting solution was allowed to cool to room temperature and diluted with ethyl acetate (200 ml). The resulting solution was washed with 2N-HCl (200 ml×2), dried over anhydrous sodium sulfate and concentrated in vacuo to afford the titled compound as an oily material.

Step 2 Preparation of 8-chloro-6-fluoro-3,4-dihydro-2H-1-benzopyran-5-amine as an Intermediate

To a solution of 8-chloro-6-fluoro-2H-1-benzopyran-5-amine (0.42 g) in ethyl acetate (16 ml) was added palladium on carbon (5%, 250 mg) was added at room temperature. The resulting suspension was stirred for 5 hours under hydrogen atmosphere and then the mixture was filtered through Celite. The filtrate was concentrated in vacuo to afford the titled compound.

¹H-NMR (CDCl₃, 400 MHz): 2.03 (2H, m), 2.51 (2H, t, J=7.0 Hz), 3.8 (2H, br s), 4.18 (2H, t, J=5.8 Hz), 6.90 (1H, d, J=10.4 Hz).

Step 3 Preparation of 8-chloro-6-fluoro-3,4-dihydro-5-isocyanato-2H-1-benzopyran as an Intermediate

To a solution of triphosgene (0.53 g) in ethyl acetate (7 ml) was added dropwise a solution of 8-chloro-6-fluoro-3,4-dihydro-2H-1-benzopyran-5-amine (0.35 g) and triethylamine (0.35 g) at 0° C. After the addition, the cold bath was removed and the resulting mixture was refluxed for one hour under nitrogen atmosphere. The mixture was allowed to cool to room temperature and white precipitate was filtered off through Celite and the filtrate was concentrated in vacuo to give the titled compound.

Step 4 To a suspension of sodium hydride (76 mg, 60% oil dispersion) in DMF (4 ml) was added dropwise a solution of ethyl 3-amino-4,4,4-trifluoromethyl crotonate (0.35 g) in toluene (3 ml) at 0° C. The resulting mixture was stirred for 30 minutes at same temperature. A solution of 8-chloro-6-fluoro-3,4-dihydro-5-isocyanato-2H-1-benzopyran in toluene (3 ml) was added and the cold bath was removed. After 2 hours, iodomethane (0.5 g) was added- and the resulting solution was stirred for additional 5 hours at ambient temperature. The resulting solution was partitioned between ethyl acetate (100 ml) and sat. brine (100 ml). The organic phase was washed with brine (100 ml×2) and dried over anhydrous sodium acetate. The solvent was removed in vacuo and the residue was subjected to column chromatography on silica gel eluting with a mixed solvent of ethyl acetate and hexane to give the titled compound (0.48 g) as a solid.

Abbreviations used in Tables are as follows.

Me: methyl, Et: ethyl, Pr: propyl, Bu: butyl, Ph: phenyl, Ac: acetyl

Representative final compounds of the present invention are set forth in the following Tables 1-7 and representative intermediate compounds of the present invention are set forth in the following Tables 8 and 9.

TABLE 1

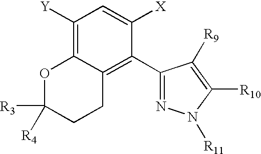

| Compd. No. | X | Y | R₃ | R₄ | R₉ | R₁₀ | R₁₁ |
|---|---|---|---|---|---|---|---|
| 1-1 | H | Cl | H | H | Cl | CF₃ | Me |
| 1-2 | F | Cl | H | H | Cl | CF₃ | Me |
| 1-3 | F | Cl | H | F | Cl | CF₃ | Me |
| 1-4 | F | Cl | H | Cl | Cl | CF₃ | Me |
| 1-5 | F | Cl | H | Br | Cl | CF₃ | Me |
| 1-6 | F | Cl | F | F | Cl | CF₃ | Me |
| 1-7 | F | Cl | Br | Br | Cl | CF₃ | Me |
| 1-8 | F | Cl | H | Me | Cl | CF₃ | Me |
| 1-9 | F | Cl | Me | Me | Cl | CF₃ | Me |
| 1-10 | F | CN | Me | Et | Cl | CF₃ | Me |
| 1-11 | H | Br | H | H | CN | CF₃ | Me |
| 1-12 | F | Cl | H | H | Cl | CHF₂ | Me |
| 1-13 | F | Cl | H | F | Cl | CHF₂ | Me |
| 1-14 | F | Cl | H | Cl | Cl | CHF₂ | Me |
| 1-15 | F | Cl | H | Br | Cl | CHF₂ | Me |
| 1-16 | F | Cl | F | F | Cl | CHF₂ | Me |
| 1-17 | F | Cl | Br | Br | Cl | CHF₂ | Me |
| 1-18 | F | Cl | H | Me | Cl | CHF₂ | Me |
| 1-19 | F | Cl | Me | Me | Cl | CHF₂ | Me |
| 1-20 | F | CN | Me | Et | Cl | CHF₂ | Me |
| 1-21 | H | Br | H | H | CN | CHF₂ | Me |
| 1-22 | F | Cl | H | H | Cl | OCF₃ | Me |
| 1-23 | F | Cl | H | F | Cl | OCF₃ | Me |
| 1-24 | F | Cl | H | Cl | Cl | OCF₃ | Me |
| 1-25 | F | Cl | H | Br | Cl | OCF₃ | Me |
| 1-26 | F | Cl | F | F | Cl | OCF₃ | Me |
| 1-27 | F | Cl | Br | Br | Cl | OCF₃ | Me |
| 1-28 | F | Cl | H | Me | Cl | OCF₃ | Me |
| 1-29 | F | Cl | Me | Me | Cl | OCF₃ | Me |
| 1-30 | F | CN | Me | Et | Cl | OCF₃ | Me |
| 1-31 | H | Br | H | H | CN | OCF₃ | Me |
| 1-32 | F | Cl | Me | Me | Cl | OCF₃ | Me |
| 1-33 | F | Cl | H | H | Cl | OCHF₂ | Me |
| 1-34 | F | Cl | H | F | Cl | OCHF₂ | Me |
| 1-35 | F | Cl | H | Cl | Cl | OCHF₂ | Me |
| 1-36 | F | Cl | H | Br | Cl | OCHF₂ | Me |
| 1-37 | F | Cl | F | F | Cl | OCHF₂ | Me |
| 1-38 | F | Cl | Br | Br | Cl | OCHF₂ | Me |
| 1-39 | F | Cl | Me | H | Cl | OCHF₂ | Me |
| 1-40 | F | Cl | Me | Me | Cl | OCHF₂ | Me |
| 1-41 | F | Cl | Me | Et | Cl | OCHF₂ | Me |
| 1-42 | F | Cl | H | Ph | Cl | OCHF₂ | Me |
| 1-43 | F | Cl | H | Et | Cl | OCHF₂ | Me |
| 1-44 | F | Cl | H | Pr | Cl | OCHF₂ | Me |
| 1-45 | F | Cl | H | Bu | Cl | OCHF₂ | Me |
| 1-46 | F | Cl | H | CH₂OH | Cl | OCHF₂ | Me |
| 1-47 | F | Cl | H | CH₂C≡CH | Cl | OCHF₂ | Me |
| 1-48 | F | Cl | H | CH₂OCOMe | Cl | OCHF₂ | Me |
| 1-49 | F | Cl | H | CH(Me)OMe | Cl | OCHF₂ | Me |
| 1-50 | F | Cl | H | CO₂Et | Cl | OCHF₂ | Me |
| 1-51 | F | Cl | H | CO₂Pr | Cl | OCHF₂ | Me |
| 1-52 | F | Cl | H | COMe | Cl | OCHF₂ | Me |
| 1-53 | F | Cl | H | CON(Et)₂ | Cl | OCHF₂ | Me |
| 1-54 | F | Cl | H | CH₂OS(O)₂Me | Cl | OCHF₂ | Me |
| 1-55 | F | Cl | H | CH═NOMe | Cl | OCHF₂ | Me |
| 1-56 | F | Cl | H | C(Me)═NOMe | Cl | OCHF₂ | Me |
| 1-57 | F | Cl | H | OCH₂COPh | Cl | OCHF₂ | Me |
| 1-58 | F | Cl | H | OSO₂Ph | Cl | OCHF₂ | Me |
| 1-59 | F | Cl | H | OCH₂CON(Me)₂ | Cl | OCHF₂ | Me |
| 1-60 | F | Cl | Me | Me | Br | OCHF₂ | Me |
| 1-61 | F | Cl | H | H | CF₃ | OCHF₂ | Me |
| 1-62 | F | Cl | H | H | Cl | OCH₂CF₃ | Me |
| 1-63 | F | Cl | H | Me | Cl | OCH₂CF₃ | Me |
| 1-64 | F | Cl | Me | Me | Cl | OCH₂CF₃ | Me |
| 1-65 | F | CN | Me | Et | Cl | OCH₂CF₃ | Me |
| 1-66 | H | Br | H | H | CN | OCH₂CF₃ | Me |
| 1-67 | F | Cl | Me | Me | Cl | OCH₂CH₂OMe | Me |
| 1-68 | F | Cl | Me | Me | Cl | CO₂Et | Me |
| 1-69 | F | Cl | H | Me | Cl | SMe | Me |
| 1-70 | F | Cl | H | Me | Cl | S(O)Me | Me |
| 1-71 | F | NO₂ | H | H | Cl | S(O)₂Me | Me |
| 1-72 | F | Cl | H | H | Cl | SCHF₂ | Me |
| 1-73 | F | Cl | H | H | Cl | S(O)CHF₂ | Me |
| 1-74 | F | Cl | —(CH₂)₂— | | Cl | CF₃ | Me |
| 1-75 | F | Cl | —(CH₂)₂— | | Cl | CHF₂ | Me |
| 1-76 | F | Cl | —(CH₂)₂— | | Cl | OCF₃ | Me |
| 1-77 | F | Cl | —(CH₂)₂— | | Cl | OCHF₂ | Me |
| 1-78 | F | Cl | —(CH₂)₂— | | Cl | OCH₂CF_{3 Me} | |
| 1-79 | F | Cl | H | H | Cl | —(CH₂)₄— | |
| 1-80 | F | Cl | H | F | Cl | —(CH₂)₄— | |
| 1-81 | F | Cl | H | Cl | Cl | —(CH₂)₄— | |
| 1-82 | F | Cl | H | Br | Cl | —(CH₂)₄— | |
| 1-83 | F | Cl | F | F | Cl | —(CH₂)₄— | |
| 1-84 | F | Cl | Br | Br | Cl | —(CH₂)₄— | |
| 1-85 | F | Cl | H | Me | Cl | —(CH₂)₄— | |
| 1-86 | F | Cl | Me | Me | Cl | —(CH₂)₄— | |
| 1-87 | F | Cl | H | CH₂C≡CH | Cl | —(CH₂)₄— | |
| 1-88 | F | Cl | H | CH(Me)OMe | Cl | —(CH₂)₄— | |
| 1-89 | F | Cl | H | CH₂OS(O)₂Me | Cl | —(CH₂)₄— | |
| 1-90 | F | Cl | H | CH═NOMe | Cl | —(CH₂)₄— | |
| 1-91 | F | Cl | H | C(Me)═NOMe | Cl | —(CH₂)₄— | |
| 1-92 | F | Cl | Me | Me | Cl | —(CH₂)₄— | |
| 1-93 | F | Cl | H | CO Me | Cl | —(CH₂)₄— | |
| 1-94 | F | Cl | H | CO N(Et)₂ | Cl | —(CH₂)₄— | |
| 1-95 | F | Cl | H | CO₂ Me | Cl | —(CH₂)₄— | |
| 1-96 | F | Cl | H | CO₂Et | Cl | —(CH₂)₄— | |
| 1-97 | F | Cl | H | OCH₂COPh | Cl | —(CH₂)₄— | |
| 1-98 | F | Cl | H | OS(O)₂Ph | Cl | —(CH₂)₄— | |
| 1-99 | F | Cl | H | OCH₂CON(Me)₂ | Cl | —(CH₂)₄— | |
| 1-100 | F | OCHF₂ | H | 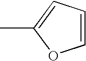 | Cl | —(CH₂)₄— | |
| 1-101 | F | Cl | H | (CH₂)₄Me | Cl | OCHF₂ | Me |
| 1-102 | F | Cl | H | (CH₂)₈Me | Cl | OCHF₂ | Me |

| | | | | | | |
|---|---|---|---|---|---|---|
| 1-103 | F | Cl | —O— | Cl | OCHF₂ | Me |

TABLE 2

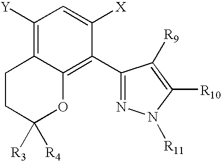

| Compd. | | | | | | | |
|---|---|---|---|---|---|---|---|
| No. | X | Y | R₃ | R₄ | R₉ | R₁₀ | R₁₁ |
| 2-1 | H | Cl | H | H | Cl | CF₃ | Me |
| 2-2 | F | Cl | H | F | Cl | CF₃ | Me |
| 2-3 | F | Cl | H | Cl | Cl | CF₃ | Me |
| 2-4 | F | Cl | H | Br | Cl | CF₃ | Me |
| 2-5 | F | Cl | F | F | Cl | CF₃ | Me |
| 2-6 | F | Cl | Br | Br | Cl | CF₃ | Me |
| 2-7 | F | Cl | H | Me | Cl | CF₃ | Me |
| 2-8 | F | Cl | Me | Me | Cl | CF₃ | Me |
| 2-9 | F | Cl | Me | Me | Cl | CHF₂ | Me |
| 2-10 | F | Cl | H | H | Cl | OCF₃ | Me |
| 2-11 | F | Cl | H | F | Cl | OCF₃ | Me |
| 2-12 | F | Cl | H | Cl | Cl | OCF₃ | Me |
| 2-13 | F | Cl | H | Br | Cl | OCF₃ | Me |
| 2-14 | F | Cl | F | F | Cl | OCF₃ | Me |
| 2-15 | F | Cl | Br | Br | Cl | OCF₃ | Me |
| 2-16 | F | Cl | H | Me | Cl | OCF₃ | Me |
| 2-17 | F | Cl | Me | Me | Cl | OCF₃ | Me |
| 2-18 | F | Cl | H | CH₂C≡CH | Cl | OCF₃ | Me |
| 2-19 | F | NO₂ | Me | CO₂Me | Cl | OCF₃ | Me |
| 2-20 | F | Cl | H | H | Br | OCHF₂ | Me |
| 2-21 | F | Cl | Me | Me | Cl | OCHF₂ | Me |
| 2-22 | H | Cl | H | H | Cl | OCHF₂ | Me |
| 2-23 | F | CN | H | H | Cl | OCHF₂ | Me |
| 2-24 | F | NO₂ | Me | CO₂Me | Cl | OCHF₂ | Me |
| 2-25 | F | Cl | H | CONMe₂ | Cl | OCHF₂ | Me |
| 2-26 | F | Cl | Me | Me | Cl | OCH₂CF₃ | Me |
| 2-27 | F | Cl | H | H | Cl | SCHF₂ | Me |
| 2-28 | F | Cl | Me | Me | Cl | S(O)₂Me | Me |

| | | | | | | |
|---|---|---|---|---|---|---|
| 2-29 | F | Cl | Me | Me | Cl | —(CH₂)₄— |
| 2-30 | F | Cl | H | CH₂C≡CH | Cl | —(CH₂)₄— |

TABLE 3

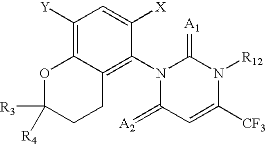

| Compd. | | | | | | | |
|---|---|---|---|---|---|---|---|
| No. | X | Y | R₃ | R₄ | R₁₂ | A₁ | A₂ |
| 3-1 | H | Cl | H | H | Me | O | O |
| 3-2 | F | Cl | H | H | Me | O | O |
| 3-3 | F | Cl | H | F | Me | O | O |
| 3-4 | F | Cl | H | Cl | Me | O | O |
| 3-5 | F | Cl | H | Br | Me | O | O |
| 3-6 | F | Cl | F | F | Me | O | O |
| 3-7 | F | Cl | Br | Br | Me | O | O |
| 3-8 | F | Cl | H | H | NH₂ | O | O |
| 3-9 | F | CN | H | Me | Me | O | O |
| 3-10 | F | NO₂ | H | Me | Me | O | O |
| 3-11 | F | Br | Me | Me | Me | O | O |
| 3-12 | F | Cl | Me | Et | Me | S | O |
| 3-13 | F | Cl | H | H | Me | S | S |
| 3-14 | F | Cl | H | H | Me | O | S |
| 3-15 | F | Cl | H | Ph | Me | O | O |
| 3-16 | F | Cl | H | Bu | Me | O | O |
| 3-17 | F | Cl | H | Pr | Me | O | O |
| 3-18 | F | H | H | H | Me | O | O |
| 3-19 | F | H | Me | CO₂NHPh | Me | O | O |
| 3-20 | F | Cl | H | CO₂Me | Me | O | O |
| 3-21 | F | Cl | H | COMe | Me | O | O |
| 3-22 | F | Cl | H | CH₂C≡CH | Me | O | O |
| 3-23 | F | Cl | H | CH(Me)OMe | Me | O | O |
| 3-24 | F | Cl | H | CH₂OS(O)₂Me | Me | O | O |
| 3-25 | F | Cl | H | CH═NOMe | Me | O | O |
| 3-26 | F | Cl | H | C(Me)═NOMe | Me | O | O |
| 3-27 | F | Cl | Me | 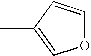 | Me | O | O |
| 3-28 | F | Cl | H | 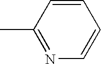 | Me | O | O |
| 3-29 | F | CN | H | CN | Me | O | O |
| 3-30 | F | CN | H | CH₂OMe | Et | O | O |

TABLE 4

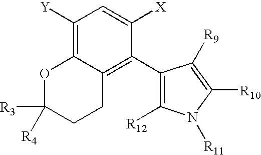

| Compd. | | | | | | | | |
|---|---|---|---|---|---|---|---|---|
| No. | X | Y | R₃ | R₄ | R₉ | R₁₀ | R₁₁ | R₁₂ |
| 4-1 | H | Cl | H | H | Cl | CF₃ | Me | Cl |
| 4-2 | F | Cl | H | H | Cl | CF₃ | Me | Cl |
| 4-3 | F | Cl | H | F | Cl | CF₃ | Me | Cl |
| 4-4 | F | Cl | H | Cl | Cl | CF₃ | Me | Cl |
| 4-5 | F | Cl | H | Br | Cl | CF₃ | Me | Cl |
| 4-6 | F | Cl | F | F | Cl | CF₃ | Me | Cl |
| 4-7 | F | Cl | Br | Br | Cl | CF₃ | Me | Cl |
| 4-8 | F | Cl | H | Me | Cl | CF₃ | Me | Cl |
| 4-9 | F | Cl | Me | Me | Cl | CF₃ | Me | Cl |
| 4-10 | F | CN | Me | Et | Cl | CF₃ | Me | Cl |
| 4-11 | H | Br | H | H | CN | CF₃ | Me | CF₃ |
| 4-12 | F | Cl | H | H | Cl | CHF₂ | Me | Cl |
| 4-13 | F | Cl | H | Me | Cl | CHF₂ | Me | Cl |
| 4-14 | F | Cl | Me | Me | Cl | CHF₂ | Me | Cl |
| 4-15 | F | Cl | H | H | Cl | OCF₃ | Me | Cl |
| 4-16 | F | Cl | H | Me | Cl | OCF₃ | Me | Cl |
| 4-17 | F | Cl | Me | Me | Cl | OCF₃ | Me | Cl |
| 4-18 | F | Cl | H | H | Cl | OCHF₂ | Me | Cl |
| 4-19 | F | Cl | Me | H | Cl | OCHF₂ | Me | Cl |
| 4-20 | F | Cl | Me | Me | Cl | OCHF₂ | Me | Cl |
| 4-21 | F | Cl | Me | Et | Cl | OCHF₂ | Me | Cl |
| 4-22 | F | Cl | H | Ph | Cl | OCHF₂ | Me | Cl |
| 4-23 | F | Cl | H | CO₂NHPh | Cl | OCHF₂ | Me | Cl |
| 4-24 | F | Cl | H | CO₂Me | Cl | OCHF₂ | Me | Cl |
| 4-25 | F | Cl | H | COMe | Cl | OCHF₂ | Me | Cl |
| 4-26 | F | Cl | H | CH₂C≡CH | Cl | OCHF₂ | Me | Cl |
| 4-27 | F | Cl | H | CH(Me)OMe | Cl | OCHF₂ | Me | Cl |
| 4-28 | F | Cl | H | CH₂OS(O)₂Me | Cl | OCHF₂ | Me | Cl |
| 4-29 | F | Cl | H | CH═NOMe | Cl | OCHF₂ | Me | Cl |
| 4-30 | F | Cl | H | C(Me)═NOMe | Cl | OCHF₂ | Me | Cl |
| 4-31 | F | Cl | H | Et | Cl | OCHF₂ | Me | H |
| 4-32 | F | Cl | H | Pr | Cl | OCHF₂ | Me | Cl |
| 4-33 | F | Cl | H | Bu | Cl | OCHF₂ | Me | Me |
| 4-34 | F | Cl | Me | Me | Br | OCHF₂ | Me | Cl |
| 4-35 | F | Cl | H | H | CF₃ | OCHF₂ | Me | Cl |
| 4-36 | F | Cl | H | Me | Cl | SMe | Me | Cl |
| 4-37 | F | Cl | H | Me | Cl | S(O)Me | Me | Cl |
| 4-38 | F | NO₂ | H | H | Cl | S(O)₂Me | Me | Cl |
| 4-39 | F | Cl | H | H | Cl | SCHF₂ | Me | Cl |
| 4-40 | F | Cl | H | H | Cl | S(O)CHF₂ | Me | Cl |
| 4-41 | F | Cl | H | H | Cl | OCF₂CF₃ | Me | OCF₂CF₃ |

| | | | | | | | |
|---|---|---|---|---|---|---|---|
| 4-42 | F | Cl | —(CH₂)₂— | Cl | OCHF₂ | Me | Cl |

| | | | | | | | |
|---|---|---|---|---|---|---|---|
| 4-43 | F | Cl | H | H | Cl | —(CH₂)₄— | Cl |
| 4-44 | F | Cl | H | Me | Cl | —(CH₂)₄— | Cl |
| 4-45 | F | Cl | Me | Me | Cl | —(CH₂)₄— | Cl |
| 4-46 | F | Cl | Me | Me | Cl | —(CH₂)₄— | Cl |
| 4-47 | F | Cl | H | CO₂Me | Cl | —(CH₂)₄— | Cl |
| 4-48 | F | OCHF₂ | H | 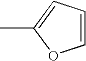 | Cl | —(CH₂)₄— | Cl |

TABLE 5

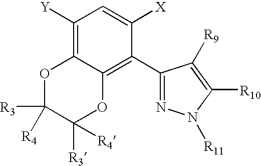

| Compd. | | | | | | | | | |
|---|---|---|---|---|---|---|---|---|---|
| No. | X | Y | R₃ | R₄ | R₃′ | R₄′ | R₉ | R₁₀ | R₁₁ |
| 5-1 | H | Cl | H | H | H | H | Cl | CF₃ | Me |
| 5-2 | F | Cl | H | H | H | H | Cl | CF₃ | Me |
| 5-3 | F | Cl | H | Me | H | Me | Cl | CF₃ | Me |
| 5-4 | F | Cl | Me | Me | H | H | Cl | CF₃ | Me |
| 5-5 | F | CN | H | H | H | H | Cl | CF₃ | Me |
| 5-6 | F | Cl | H | H | H | H | Cl | CHF₂ | Me |
| 5-7 | F | Cl | H | Me | H | Me | Cl | CHF₂ | Me |
| 5-8 | F | Cl | Me | Me | H | H | Cl | CHF₂ | Me |
| 5-9 | F | Cl | H | H | H | H | Cl | OCF₃ | Me |
| 5-10 | F | Cl | H | Me | H | Me | Cl | OCF₃ | Me |
| 5-11 | F | Cl | Me | Me | H | H | Cl | OCF₃ | Me |
| 5-12 | F | Cl | H | H | H | H | Br | OCHF₂ | Me |
| 5-13 | F | Cl | H | Me | H | Me | Cl | OCHF₂ | Me |
| 5-14 | F | Cl | Me | Me | H | H | Cl | OCHF₂ | Me |
| 5-15 | F | Cl | H | H | H | H | Cl | SCHF₂ | Me |
| 5-16 | F | Cl | H | H | H | H | CN | S(O)CHF₂ | Me |
| 5-17 | F | Cl | H | Me | H | Me | Cl | OMe | Me |
| 5-18 | F | Cl | H | H | —O— | | Cl | OCHF₂ | Me |
| 5-19 | F | Cl | H | H | H | H | Cl | —(CH₂)₄— | |
| 5-20 | F | Cl | H | Bu | H | H | Cl | OCHF₂ | Me |

TABLE 6

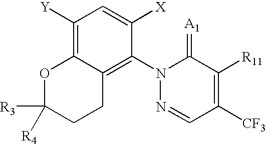

| Compd. | | | | | | |
|---|---|---|---|---|---|---|
| No. | X | Y | R₃ | R₄ | R₁₁ | A₁ |
| 6-1 | F | Cl | H | H | Me | O |
| 6-2 | F | Cl | H | H | Me | O |
| 6-3 | F | Cl | H | H | Me | O |
| 6-4 | F | Cl | H | F | Me | O |
| 6-5 | F | Cl | H | Cl | Me | O |
| 6-6 | F | Cl | F | Br | Me | O |
| 6-7 | F | Cl | Br | F | Me | O |
| 6-8 | F | Cl | H | Br | Me | O |
| 6-9 | F | Cl | Me | Me | Me | O |
| 6-10 | F | Cl | H | Me | Me | O |
| 6-11 | F | Cl | H | Me | Me | S |
| 6-12 | F | CN | Me | H | Me | O |
| 6-13 | F | Cl | H | Me | Me | O |
| 6-14 | F | Cl | H | Me | NH₂ | O |
| 6-15 | F | Cl | H | Bu | Me | O |
| 6-16 | F | Cl | H | Ph | Me | O |
| 6-17 | F | Cl | H | COMe | Me | O |
| 6-18 | F | Cl | H | CON(Et)₂ | Me | O |
| 6-19 | F | Cl | H | CH₂OH | Me | O |
| 6-20 | F | Cl | H | CH₂C≡CH | Me | O |
| 6-21 | F | Cl | H | CH₂OCOMe | Me | O |
| 6-22 | F | Cl | H | CH(Me)OMe | Me | O |
| 6-23 | F | Cl | H | CO₂Et | Me | O |
| 6-24 | F | Cl | H | CO₂Pr | Me | O |
| 6-25 | F | Cl | H | CO₂Me | Me | O |
| 6-26 | F | Cl | H | CH₂OS(O)₂Me | Me | O |
| 6-27 | F | Cl | H | CH═NOMe | Me | O |
| 6-28 | F | Cl | H | C(Me)═NOMe | Me | O |
| 6-29 | F | Cl | H | OCH₂COPh | Me | O |
| 6-30 | F | Cl | H | OS(O)₂Ph | Me | O |
| 6-31 | F | Cl | H | OCH₂CON(Me)₂ | Me | O |

TABLE 7

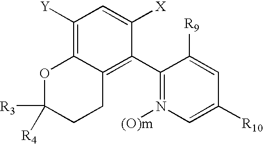

| Compd. | | | | | | | |
|---|---|---|---|---|---|---|---|
| No. | X | Y | R₃ | R₄ | R₉ | R₁₀ | m |
| 7-1 | H | Cl | H | H | Cl | CF₃ | 0 |
| 7-2 | F | Cl | H | H | Cl | CF₃ | 0 |
| 7-3 | F | Cl | H | H | F | CF₃ | 0 |
| 7-4 | F | Cl | H | Me | Cl | CF₃ | 0 |
| 7-5 | F | Cl | Me | Me | Cl | CF₃ | 0 |
| 7-6 | F | CL | Me | Me | F | CF₃ | 0 |
| 7-7 | F | CN | Me | Et | Cl | CF₃ | 0 |
| 7-8 | H | Br | H | H | CN | CF₃ | 0 |
| 7-9 | F | Cl | H | H | Cl | CF₃ | 1 |
| 7-10 | F | Cl | Me | H | Cl | CF₃ | 1 |
| 7-11 | F | Cl | Me | Me | Cl | CF₃ | 1 |
| 7-12 | F | Cl | Me | Et | Cl | CF₃ | 0 |
| 7-13 | F | Cl | H | Ph | Cl | CF₃ | 0 |
| 7-14 | F | Cl | H | Et | Cl | CF₃ | 0 |
| 7-15 | F | Cl | H | Pr | Cl | CF₃ | 0 |
| 7-16 | F | Cl | H | Bu | Cl | CF₃ | 0 |
| 7-17 | F | Cl | Me | Me | Br | CF₃ | 0 |
| 7-18 | F | Cl | H | H | CF | CF₃ | 0 |
| 7-19 | F | Cl | H | Me | F | CF₃ | 0 |
| 7-20 | F | Cl | H | Me | F | CF₃ | 1 |
| 7-21 | F | NO₂ | H | H | Cl | CF₃ | 0 |
| 7-22 | F | Cl | H | H | Cl | OCHF₂ | 0 |
| 7-23 | F | Cl | H | H | Cl | SCHF₂ | 0 |
| 7-24 | F | Cl | H | H | Cl | S(O)Me | 0 |
| 7-25 | F | Cl | —(CH₂)₂— | | Cl | CF₃ | 0 |
| 7-26 | F | Cl | H | H | Cl | SMe | 0 |
| 7-27 | F | Cl | H | Me | Cl | S(O)CHF₂ | 0 |
| 7-28 | F | Cl | Me | Me | Cl | OCF₃ | 0 |
| 7-29 | F | Cl | Me | Me | F | OCHF₂ | 0 |
| 7-30 | F | Cl | H | CO₂Me | Cl | CF₃ | 0 |
| 7-31 | F | Cl | H | CON(Et)₂ | Cl | CF₃ | 0 |
| 7-32 | F | Cl | H | CH₂OH | Cl | CF₃ | 0 |
| 7-33 | F | Cl | H | CH₂C═CH | Cl | CF₃ | 0 |
| 7-34 | F | Cl | H | CH₂OCOMe | Cl | CF₃ | 0 |
| 7-35 | F | Cl | H | CH(Me)OMe | Cl | CF₃ | 0 |
| 7-36 | F | Cl | H | CO₂Et | Cl | CF₃ | 0 |
| 7-37 | F | Cl | H | CO₂Pr | Cl | CF₃ | 0 |
| 7-38 | F | Cl | H | COMe | Cl | CF₃ | 0 |
| 7-39 | F | Cl | H | CH₂OS(O)₂Me | Cl | CF₃ | 0 |
| 7-40 | F | Cl | H | CH═NOMe | Cl | CF₃ | 0 |
| 7-41 | F | Cl | H | C(Me)═NOMe | Cl | CF₃ | 0 |
| 7-42 | F | Cl | H | OCH₂COPh | Cl | CF₃ | 0 |
| 7-43 | F | Cl | H | OS(O)₂Ph | Cl | CF₃ | 0 |
| 7-44 | F | Cl | H | OCH₂CON(Me)₂ | Cl | CF₃ | 0 |
| 7-45 | F | OCHF₂ | H | 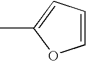 | Cl | CF₃ | 0 |
| 7-46 | F | Br | H | COMe | Cl | CF₃ | 0 |
| 7-47 | F | Cl | H | 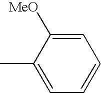 | Cl | CF₃ | 0 |
| 7-48 | F | Cl | H | CH₂Cl | Cl | CF₃ | 0 |

TABLE 8

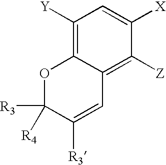

| Compd. | | | | | | |
|---|---|---|---|---|---|---|
| No. | X | Y | R₃ | R₄ | R₃′ | Z |
| 8-1 | H | Cl | H | H | H | NH₂ |
| 8-2 | F | Cl | H | H | H | NO₂ |
| 8-3 | F | Br | H | H | H | NH₂ |
| 8-4 | F | CN | H | H | H | NH₂ |
| 8-5 | F | Cl | H | Me | H | NH₂ |
| 8-6 | F | Cl | H | H | H | CO₂H |
| 8-7 | F | Cl | H | H | H | COCl |
| 8-8 | F | Cl | Me | Me | H | NH₂ |
| 8-9 | F | Cl | H | Me | F | NH₂ |
| 8-10 | Cl | Cl | H | H | H | CO₂H |
| 8-11 | H | NO₂ | H | Et | H | NH₂ |
| 8-12 | F | Cl | H | Bu | H | NH₂ |
| 8-13 | F | Cl | H | Bu | Me | NH₂ |
| 8-14 | F | Cl | H | Me | CO₂Me | NH₂ |
| 8-15 | F | Cl | H | (CH₂)₈Me | H | NH₂ |
| 8-16 | F | Cl | H | Me | H | Br |
| 8-17 | F | Cl | H | H | H | Me |
| 8-18 | F | Cl | H | Me | H | Ac |
| 8-19 | F | Cl | Me | Me | H | Ac |

TABLE 9

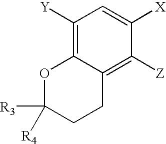

| Compd. No. | X | Y | R₃ | R₄ | Z |
|---|---|---|---|---|---|
| 9-1 | H | Cl | H | H | NH₂ |
| 9-2 | F | Cl | H | H | NO₂ |
| 9-3 | F | Br | H | H | NH₂ |
| 9-4 | F | CN | H | H | NH₂ |
| 9-5 | F | Cl | H | Me | NH₂ |
| 9-6 | F | Cl | H | H | CO₂H |
| 9-7 | F | Cl | H | H | COCl |
| 9-8 | F | Cl | Me | Me | NH₂ |
| 9-9 | F | Cl | H | Me | NH₂ |
| 9-10 | Cl | Cl | H | H | CO₂H |
| 9-11 | H | NO₂ | H | Et | NH₂ |
| 9-12 | F | Cl | H | Bu | NH₂ |
| 9-13 | F | Cl | H | Bu | NH₂ |
| 9-14 | F | Cl | H | Me | NH₂ |
| 9-15 | F | Cl | H | (CH₂)₈Me | NH₂ |
| 9-16 | F | Cl | H | Me | Br |
| 9-17 | F | Cl | H | H | Me |
| 9-18 | F | Cl | Me | Me | Ac |

Table 10 listed some of the characterization data for the representative compounds of this invention.

TABLE 10

| Compd. | |
|---|---|
| No. | ¹H-NMR |
| 1-2 | 1.96(2H, m), 2.59(2H, br s), 4.05(3H, s), 4.27(2H, t, J = 5.1 Hz), 7.09(1H, |
| | d, J = 8.6 Hz). |
| 1-8 | 1.38(3H, d, J = 6.0 Hz), 1.61(1H, m), 1.88(1H, m), 2.4-2.6(2H, m), |
| | 3.98(3H, s), 4.13(1H, m), 7.00(1H, d, J = 8.8 Hz). |
| 1-33 | 1.96(2H, m), 2.63(2H, m), 3.83(3H, s), 4.26(2H, t, J = 5.2 Hz), 6.72(1H, t, |
| | J = 72.3 Hz), 7.07(1H, d, J = 8.7 Hz). |
| 1-39 | 1.38(3H, d, J = 6.4 Hz), 1.60(1H, m), 1.92(1H, m), 2.55(1H, m), 2.70(1H, |
| | m), 3.80(3H, s), 4.20(1H, dqd, J = 10.4, 6.4, 2.4 Hz), 6.70(1H, t, |
| | J = 72.4 Hz), 7.05(1H, d, J = 8.4 Hz). |
| 1-40 | 1.37(6H, s), 1.75(2H, t, J = 6.8 Hz), 2.62(2H, t, J = 6.8 Hz), 3.83(1H, s), |
| | 6.72(1H, t, J = 72.6 Hz), 7.10(1H, d, J = 8.8 Hz). |
| 1-42 | 1.99(1H, m), 2.19(1H, m), 2.65(1H, m), 2.80(1H, m), 3.82(3H, s), |
| | 5.14(1H, m), 6.69(1H, t, J = 72.6 Hz), 7.10(1H, J = 8.8 Hz), 7.3-7.5(5H, m). |
| 1-72 | 1.96(2H, m), 2.59(2H, m), 4.05(3H, s), 4.27(2H, t, J = 5.2 Hz), 6.78(1H, t, |
| | J = 56.4 Hz), 7.08(1H, d, J = 8.8 Hz). |
| 1-73 | 1.95(2H, m), 2.57(2H, m), 4.16(3H, s), 4.26(2H, t, J = 5.2 Hz), 6.72(1H, t, |
| | J = 54.4 Hz), 7.07(1H, d, J = 8.8 Hz). |
| 1-79 | 1.93(4H, m), 2.07(2H, m), 2.65(2H, br s), 2.77(2H, t, J = 6.3 Hz), 4.15(2H, |
| | t, J = 6.3 Hz), 4.25(2H, t, J = 5.0), 7.05(1H, d, J = 8.8 Hz). |
| 1-85 | 1.42(3H, d, J = 6.4 Hz), 1.61(1H, m), 1.91(3H, m), 2.06(2H, m), 2.5-2.7(2H, |
| | m), 2.75(2H, t, J = 6.4 Hz), 4.14(2H, t, J = 5.8 Hz), 4.18(1H, m), 7.02(1H, d, |
| | J = 8.8 Hz). |
| 1-101 | 0.80(3H, m), 1.2-1.6(8H, m), 1.75(1H, m), 1.90(1H, m), 2.50(1H, m), |
| | 2.60(1H, m), 3.75(3H, s) 3.95(1H, m), 6.64(1H, t, J = 72.4 Hz), 6.98(1H, d, |
| | J = 8.8 Hz). |
| 3-2 | 1.98(2H, quin., J = 6.4 Hz), 2.50(2H, t, J = 6.5 Hz), 3.54(3H, s), 4.25(2H, t, |
| | J = 5.2 Hz), 6.34(1H, s), 7.13(1H, d, J = 8.8 Hz). |
| 7-2 | 1.90(2H, m), 2.17(1H, dt, J = 17.2, 5.6 Hz), 2.57(1H, dt, J = 17.2, 6.8 Hz), |
| | 4.22(2H, m), 7.04(1H, d, J = 8.4 Hz), 8.01(1H, s), 8.80(1H, s). |
| 7-4 | 1.43(3H, m), 1.5-2.7(4H, m), 4.20(1H, m), 7.08(1H, d, J = 8.8 Hz), |
| | 8.05(1H, s), 8.85(1H, br s). |

Herbicidal Activity

The compounds of the present invention exhibit excellent herbicidal effect when used as an active ingredient of a herbicide. The herbicide can be used for a wide range of applications, for example on croplands such as paddy fields, upland fields, orchards vineyards and mulberry fields, and non-crop lands such as forests, turf, right of way, roadsides, railroads, playgrounds and factory sites. The application method may be suitable selected for soil treatment application, foliar application and water application.

The compounds of the present invention are capable of controlling noxious weeds including grass (gramineae) such as barnyardgrass (Echinochloa crus-galli L.), crabgrass (Digitaria sanguinalis L.), green foxtail (Setaria viridis L.), goosegrass (Eleusine indica L.), wild oat (Avena fatua L.), johnsongrass (Sorghum halepense L.), quackgrass (Agropyron repens L.), alexandergrass (Brachiaria plantaginea), paragrass (Panicum purpurascens), sprangletop (Leptochloa chinensis) and red sprangletop (Leptochloa panicea); sedges (Cyperaceae) such as rice flatsedge (Cyperus iria L.), purple nutsedge (Cyperus rotundus L.), japanese bulrush (Scirpus juncoides), flatsedge (Cyperus serotinus), smallflower umbrellaplant (Cyperus difformis L.), slender spikerush (Eleocharis acicularis L.), and water chestnut (Eleocharis kuroguwai); alismataceae such as japanese ribbon wapato (Sagittaria pygmaea), arrow-head (Sagittaria trifolia L.) and narrowleaf waterplantain (Alisma canaliculatum); pontederiaceae such as monochoria (Monochoria vaginalis) and monochoria species (Monochoria korsakowii); scrophulariaceae such as falsepimpemel (Lindernia pyxidaria) and dopatrium (Dopatrium junceum); lythraceae such as toothcup (Rotala indica) and red stem (Ammannia multiflora), and broadleaves such as redroot pigweed (Amaranthus retroflexus L.), velvetleaf (Abutilon theophrasti), morningglory (Ipomoea hederacea L.), lambsquaters (Chenopodium album L.), prickly sida (Sida spinosa L.), common purslane (Portulaca oleracea L.), slender amaranth (Amaranthus viridis L.), sicklepod (Cassia obtusifolia L.), black nightshade (Solanum nigrum L.), pale smartweed (Polygonum lapathifolium L.), common chcikweed (Stellaria media L.), common cocklebur (Xanthium strumarium L.), flexuous bittercress (Cardamine flexuosa), henbit (Lamium amplexicaule L.) and threeseeded copperleaf (Acalypha australis L.). Accordingly, it is useful for controlling noxious weeds non-selectively or selectively in the cultivation of a crop plant such as corn (Zea mays L.), soybean (Glycine max Merr.), cotton (Gossypium spp.), wheat (Triticum spp.), rice (Oryza sativa L.), barley (Hordeum vulgare L.), oats (Avena sativa L.), sorgo (Sorghum bicolor Moench), rape (Brassica napus L., Brassica campestris L.), sunflower (Helianthus annuus L.), sugar beet (Beta vulgaris L.), sugar cane (Saccharum officinarum L.), japanese lawngrass (Zoysia japonica Steud), peanut (Arachis hypogaea L.) or flax (Linum usitatissimum L.). The compound of the present invention is particularly effective for selectively controlling noxious weeds in the cultivation of corn, wheat, rice, soybean or cotton, especially in the cultivation of corn, soybean, wheat and rice.

For the use as herbicides, the active ingredients of this invention are formulated into herbicidal compositions by mixing herbicidal active amounts with inert ingredients known to the art to facilitate either the suspension, dissolution or emulsification of the active ingredient for the desired use. The type of formulation prepared recognizes the facts that formulation, crop and use pattern all can influence the activity and utility of active ingredient in a particular use. Thus for agricultural use the present herbicidal compounds may be formulated as water dispersible granules, granules, suspension concentrates, tablets, capsules, wettable powders, dusts, solutions, emulsifiable concentrates (EC), microemulsion, suspoemulsion, invert emulsion or other type of formulations, depending on the desired weed targets, crops and application methods. However, so long as it is suitable for the purpose of the present invention, it may be formulated into any type of formulation that is commonly used in this field.

These herbicidal formulations may be applied to the target area (Where suppression of unwanted vegetation is the objective) as dusts, granules, tablets, capsules or water or solvent diluted sprays. These formulation may be contain as little as 0.1% to as much as 97% active ingredient by weight.

Dusts are admixtures of the active ingredient with finely ground materials such as clays (some examples include kaolin and montmorillonite clays), talc, granite dust or other organic or inorganic solids which act as dispersants and carriers for the active ingredient; these finely ground materials have an average particle size of less than 50 microns. A typical dust formulation will contain 1% active ingredient and 99% carrier.

Wettable powders are composed of finely ground particles which disperse rapidly in water or other spray carriers. Typical carriers include kaolin clays, fullers earth, silicas and other absorbent, wettable inorganic materials. Wettable powders can be prepared to contain from 0.1 to 90% active ingredient, depending on the desired use pattern and the absorbability of the carrier. Wettable powders typically contain wetting or dispersing agent to assist dispersion in water or other carriers.

Water dispersible granules are granulated solids that freely disperse when mixed in water. This formulation typically consists of the active ingredient (0.1% to 95% active ingredient), a wetting agent (1 to 15% by weight), a dispersing agent (1 to 15% by weight) and an inert carrier (1 to 95% by weight). Water dispersible granules can be formed by mixing the ingredients intimately then adding a small amount of water on a rotating disc (said mechanism is commercially available) and collecting the agglomerated granules. Alternatively, the mixture of ingredients may be mixed with an optimal amount of liquid (water or other liquid) and passed through an extruder (said mechanism is commercially available) equipped with passages which allow for the formulation of small extruded granules. Alternatively, the mixture of ingredients can be granulated using a high speed mixer (said mechanism is commercially available) by adding a small amount of liquid and mixing at high speeds to affect agglomeration. Alternatively, the mixture of ingredient can be dispersed in water and dried by spraying the dispersion through a heated nozzle in a process known as spray drying (spray drying equipment is commercially available). After granulation the moisture content of granules is adjusted to an optimal level (generally less than 5%) and the product is sized to the desired mesh size.

Granules are granulated solids that do not disperse readily in water, but instead maintain their physical structure when applied to the soil or water using a dry granule applicator or by hand. These granulated solids may be made of clay, vegetable material such as corn cob grids, agglomerated silicas or other agglomerated organic or inorganic materials or compounds such as calcium sulfate. The formulation typically consists of the active ingredient (1 to 20%) dispersed on or absorbed into the granule. The granule may be produced by intimately mixing the active ingredient with the granules with or without a sticking agent to facilitate adhesion of the active ingredient to the granule surface, or by dissolving the active ingredient in a solvent, spraying the dissolved active ingredient and solvent onto the granule then drying to remove the solvent. Granular formulations are useful where in-furrow or banded application is desired.

Emulsifiable concentrates (EC) are homogeneous liquids composed of a solvent or mixture of solvent such as xylenes, heavy aromatic naphthas, isophorone or other proprietary commercial compositions derived from petroleum distillates, the active ingredient and an emulsifying agent or agents. For herbicidal use, the EC is added to water (or other spray carrier) and applied as a spray to the target area. The composition of an EC formulation can contain 0.1% to 95% active ingredient, 4 to 95% solvent or solvent mixture and 1 to 20% emulsifying agent or mixture of emulsifying agent.

Suspension concentrate (also known as flowable) formulations are liquid formulations consisting of a finely ground suspension of the active ingredient in a carrier, typically water or a non-aqueous carrier such as an oil. Suspension concentrates typically contain the active ingredient (0.1 to 50% by weight), carrier, wetting agent, dispersing agent, anti-freeze, viscosity modifiers and pH modifiers. For application, suspension concentrates are typically diluted with water and sprayed on the target area or they are directly applied to flooded water in paddy field.

Microemulsions are solutions consisting of the active ingredient (1 to 30%) dissolved in a surfactant or emulsifier, with additional solvents. Microemulsions are particularly useful when a low odor formulation is required such as in residential turfgrass applications.

Suspoemulsions are combinations of two active ingredients. One active ingredient is made as a suspension concentrate (1 to 50% active ingredient) and the second active ingredient is made as an emulsifiable concentrate (0.1 to 20%). A reason for making this kind of formulation is the inability to make an EC formulation of the first ingredient due to poor solubility in organic solvents. The suspoemulsion formulation allows for the combination of the two active ingredients to be packaged in one container, thereby minimizing packaging waste and giving greater convenience to the product user.

The herbicidal compounds of this invention may be formulated or applied with insecticides, fungicides, acaricides, nematicides, fertilizers, plant growth regulators or other agricultural chemicals. Certain tank mix additives, such as spreader stickers, penetration aids, wetting agents, surfactants, emulsifiers, humectants and UV protectants may be added in amount of 0.01% to 5% to enhance the biological activity, stability, wetting, spreading on foliage or uptake of active ingredients on the target area or to improve the suspensibility, dispersion, redispersion, emulsifiability, UV stability or other physical or physico-chemical property of the active ingredient in the spray tank, spray system or target area.

The dose of the herbicidal composition of the present invention can not generally be defined, since it may be vary depending upon the weather condition, the soil condition, the type of the formulation, the types of the weeds to controlled, season for application, etc. However, it is usually applied so that the compound of the present invention would be applied in an amount of from 0.5 to 5000 g/ha, preferably from 1 to 1000 g/ha, more preferably from 5 to 500 g/ha. The present invention covers such a method for controlling noxious weeds by application of such a herbicidal composition.

The herbicidal compositions of the present invention may be used in admixture with or in combination with other agricultural chemicals, fertilizers, adjuvants, surfactants, emulsifiers, oils, polymers and phytotoxicity-reducing agent such as herbicide safeners. In such a case, they may exhibit even better effects or activities. As other agricultural chemicals, herbicides, fungicides, antibiotics, plant hormones, plant growth regulators, insecticides or acaricides may, for example, be mentioned. Especially with herbicidal compositions having the compounds of the present invention used in admixture with or in combination with one or more active ingredients of other herbicides, it is possible to improve the herbicidal activities, the range of application time(s) and the range of applicable weed types. Further, the compounds of the present invention and an active ingredient of another herbicide may be separately formulated so they may be mixed for use at the time of application, or both may be formulated together. The present invention covers such herbicidal compositions.

The blend ratio of the compounds of the present invention with the active ingredient of other herbicides can not generally be defined, since it varies depending on the time and method of application, weather conditions, soil type and type of formulation, etc. However one active ingredient of other herbicide may be incorporated usually in an amount of 0.001 to 10000 parts by weight, preferably from 0.01 to 1000 parts by weight, per one part by weight of compounds of present invention. Further, the total dose of all of the active ingredients is usually from 0.1 to 10000 g/ha, preferably from 0.2 to 5000 g/ha. The present invention covers a method for controlling noxious weeds by application of such herbicidal compositions.

As the active ingredient of the other herbicides, non-limiting examples of which are mentioned below (common name). Herbicidal compositions having the compounds of the present invention used in combination with other herbicides may be occasionally exhibit a synergistic effect.

1. Those that are believed to exhibit herbicidal effects by disturbing hormone activities of plants, including a phenoxy acetic acid type such as 2,4-D, 2,4-DB, 2,4-DP, MCPA, MCPP, MCPB or naproanilide (including the free acids, esters or salts thereof), an aromatic carboxylic type such as 2,3,6 TBA, dicamba or dichlobenil, a pyridine type such as picloram (including the free acids and salts thereof), triclopyr or clopyralid and others such as naptalam, benazolin, quinclorac, quinmerac, diflufenzopyr or thiazopyr.

2. Those that are believed to exhibit herbicidal effects by inhibiting photosynthesis of plants including a urea type such as diuron, linuron, isoproturon, chlorotoluron, metobenzuron, tebuthiuron or fluometuron, a triazine type such as simazine, atrazine, cyanazine, terbuthylazine, atraton, hexazinone, metribuzin, simetryn, prometryn, dimethametryn, triaziflam, propazine or ametryn, uracil type such as bromacil, terbacil or lenacil, an anilide type such as propanil or cypromid, a carbamate type such as swep, desmedipham or phenmedipham, a hydroxybenzonitrile type such as bromoxynil, bromoxynil-octanoate or ioxynil, and others such as pyridate, bentazon, amicarbazone or methazole.

3. A quaternary ammonium salt type such as paraquat, diquat or difenzoquat, which is believed to form active oxygen in the plant and thus to exhibit quick herbicidal effects.

4. Those which are believed to exhibit herbicidal effects by inhibiting chlorophyll biosynthesis in the plant body and abnormally accumulating a photosensitizing peroxide substance in the plant body, including a diphenyl ether type such as nitrofen, lactofen, ethoxyfen-ethyl, acifluorfen-sodium, oxyfluorfen, fomesafen, bifenox or chlomethoxyfen, a cyclic imide type such as chlorphthalim, flumioxazin, cinidon-ethyl or flumiclorac-pentyl, and others such as oxadiazon, sulfentrazone, thidiazimin, azafenidin, carfentrazone-ethyl, isopropazole, fluthiacet-methyl, pentoxazone, pyraflufen-ethyl, benzfendizone, butafenacil, metobenzuron, flupoxam, fluazolate, profluazol, pyrachlonil or oxadiargyl.

5. Those which are believed to exhibit herbicidal effects characterized by whitening activities by inhibiting chromogenesis of plants such as carotenoids including a pyridazinone type such as norflurazon, chloridazon or metflurazon, a pyrazol type such as pyrazolate, pyrazoxyfen or benzofenap, and others such as fluridone, flurtamone, diflufenican, methoxyphenone, clomazone, amitrole, sulcotrione, mesotrione, isoxaflutole, isoxachlortole, bezobicyclon, picolinafen or beflubutamid.

6. Those which exhibit herbicidal effects specifically to gramineous plants including an aryloxyphenoxypropoinic acid type (either as a mixture of isomers or as a resolved isomer) such as diclofop-methyl, pyriphenop-sodium, fluazifop-butyl, haloxyfop-methyl, quizalofop-ethyl, fenoxaprop-ethyl, flamprop-methyl or cyhalofop-butyl, and a cyclohexanedione type such as alloxydim-sodium, clethodim, tepraloxydim, tralkoxydim, butroxydim, caloxydim, clefoxydim or sethoxydim.

7. Those which are believed to exhibit herbicidal effects by inhibiting amino acid biosynthesis of plants, including a sulfonylurea type such chlorimuron-ethyl, nicosulfuron, metsulfuron-methyl, triasulfuron, primisulfuron, tribenuron-methyl, chlorsulfuron, bensulfuron-methyl, sulfometuron-methyl, prosulfuron, halosulfuron, halosulfuron-methyl, thifensulfuron-methyl, rimsulfuron, azimsulfuron, flazasulfuron, imazosulfuron, cyclosulfamuron, flupyrsulfuron, iodosulfuron, ethoxysulfuron, cinosulfuron, pyrazosulfuron-ethyl, trisulfuron-methyl, ethametsulfuron, flupyrsulfuron, tritosulfuron, foramsulfuron, trifloxysulfuron, sulfosulfuron or oxasulfuron, and a triazolopyrimidinesulfonamide type such as flumetsulam, metosulam, chloransulam, chloransulam-methyl, diclosulam, florasulam, metosulfam or penoxsulam, an imidazolinone type such as imazapyr, imazethapyr, imazaquin, imazamox, imazameth, imazapic or imazamethabenz-methyl, a pyrimidinesalicylic acid type such as pyrithiobac-sodium, bispyribac-sodium, pyriminobac-methyl, pyribenzoxim or pyriftalid, a sulfonylaminocarbonyltriazolinone type such as flucarbazone-sodium or procarbazone-sodium (MKH6561), and others such as glyphosate, glyphosate-ammonium, glyphosate-isopropylamine, sulfosate, glufosinate, glufosinate-ammonium, phosphinothricin or bialaphos.

8. Those which are believed to exhibit herbicidal effects by inhibiting cell division of plant cell, including a dinitroaniline type such as trifluralin, oryzalin, nitralin, pendimethalin, ethafluralin, benefin or prodiamine, an amide type such as bensulide, napronamide or pronamide, a carbamate type such as propham, chlorpropham, barban or asulam, a cumylamine type such as daimuron, cumyluron or bromobutide, an organophosphorous type such as amiprofos-methyl, butamifos, anilofos or piperophos, and others such as DCPA, dithiopyr, asulam or thiazopyr.

9. Those which are believed to exhibit herbicidal effects by inhibiting lipid synthesis of plant cells, including a chloroacetanilide type such as alachlor, metolachlor (including combinations with safeners such as benoxacor, or resolved isomeric mixtures of metolachlor including safeners such as nenoxacor), propachlor, acetochlor (including combinations with herbicide safeners such as dichiomid or MON-4660, or resolved isomeric mixtures of acetochlor containing safeners such as dichlomid or MON-4660), propisochlor, butachlor, pretilachlor, thenylchlor, pethoxamide or dimethenamid, an oxyacetamide type such as flufenact or mefenacet, and others such as etobenzanid, tridiphane, cafenstrol, fentrazamide, oxaziclomefone or indanofan.

10. Those in which the mode of action causing the herbicidal effects are not well understood including the thiocarbamates such as thiobencarb, EPTC, diallate, triallate, molinate, pebulate, cycloate, butylate, vemolate, prosulfocarb, dimepiperate, fenazolate, esprocarb or pyributicarb, and miscellaneous herbicides such as MSMA, DSMA, endothall, ethofumesate, sodium chlorate, pelargonic acid or fosamine.

11. Those which are believed to exhibit herbicidal effects by infestation on the plant bodies, including a biological herbicide such as Xanthomonas campestris, Epicoccosurus nematosurus, Exserohilum monoseras, Drechsrela monoceras.

A few formulation examples of the present invention are given as follows.

### FORMULATION EXAMPLE 1

### Water-Dispersible Granule

| | | | | % |
|---|---|---|---|---|
| | Trade Name | Chemical Name | Supplier | wt./wt |
| (1) | Compound of | | | 75 |
| | this invention | | | |
| (2) | Geropon T-77 | Sodium N-methyl- | Rhone-Poulenc | 14.5 |
| | | N-oleoyl taurate | | |
| (3) | NaCl | | | 10 |
| (4) | Dextrin | | | 0.5 |

The above pulverized components are placed in a high-speed mixing granular, admixed with 20 wt % of water, granulated, and dried to form water-dispersible granules.

### FORMULATION EXAMPLE 2

### Wettable Powder

| | | | | % |
|---|---|---|---|---|
| | Trade Name | Chemical Name | Supplier | wt./wt |
| (1) | Compound of | | | 10 |
| | this invention | | | |
| (2) | Kaolin clay | | | 70.2 |
| (3) | Laveline FAN | Condensate of sodium | Dai-ichi Kogyo | 1.8 |
| | | napthalene sulfate | Seiyaku co., | |
| | | and formalin | Ltd. | |
| (4) | Sorpol 5039 | Sodium polyoxyethlene | Toho Chemical | 4.5 |
| | | alkylaryl ether sulfate- | Industry Co., | |
| | | premix with white carbon | Ltd. | |
| (5) | Carplex | White carbon | Shionogi | 13.5 |
| | | | Seiyaku | |
| | | | Co., Ltd. | |

The above pulverized components (2) to (5) and compound (1) are mixed to obtain a wettable powder.

### FORMULATION EXAMPLE 3

### Wettable Powder

| | | | | % |
|---|---|---|---|---|
| | Trade Name | Chemical Name | Supplier | wt./wt |
| (1) | Compound of | | | 60 |
| | this | | | |
| | invention | | | |
| (2) | Hi-Filler | Talc micropowder | Matsumura | 33 |
| | No. 10 | | Sangyo | |
| | | | Co., Ltd. | |
| (3) | Sorpol 5050 | Dialkyl sulsosuccinate- | Toho Chemical | 3 |
| | | premixed with white | Industry Co., | |
| | | carbon | Ltd. | |
| (4) | Sorpol 5073 | A mixture of | Toho Chemical | 4 |
| | | polyoxyethlene | Industry Co., | |
| | | alkylaryl ether sulfate | Ltd. | |
| | | and a polyoxyethylene | | |
| | | monomethyl ether | | |
| | | carbonate, premixed | | |
| | | with white carbon | | |

The above pulverized compound (1) and the components (2) to (4) are mixed to obtain a wettable powder.

### FORMULATION EXAMPLE 4

### Granule

| | | | | % |
|---|---|---|---|---|
| | Trade Name | Chemical Name | Supplier | wt./wt |
| (1) | Compound of | | | 4 |
| | this invention | | | |
| (2) | Bentonite | | | 30 |
| (3) | Calcium carbonate | | | 61.5 |
| (4) | Toxanon GR-31A | Polycarboxylic | Sanyo | 3 |
| | | acid type | Chemical | |
| | | surfactant | Industries | |
| | | | Co., Ltd. | |
| (5) | Calcium lignin | | | 1.5 |
| | sulfonate | | | |

Pulverized compound (1), components (2) and (3) are preliminarily mixed, and then components (4), (5) and water are mixed thereto. The mixture is extruded and granulated, followed by drying and size-adjusting to obtain granules.

### FORMULATION EXAMPLE 5

### Water-Dispersible Granule

| | | | | % |
|---|---|---|---|---|
| | Trade Name | Chemical Name | Supplier | wt./wt |
| (1) | Compound of | | | 30 |
| | this invention | | | |
| (2) | Zieclite | A pulveried product | Zieclite | 60 |
| | | of a mixture of | Co., Ltd. | |
| | | kaolinite and | | |
| | | sericite | | |
| (3) | New Kalgen | Alkyl naphalene | Takemoto | 5 |
| | WG-1 | sulfonate | Oil and Fats | |
| | | | Co., Ltd. | |
| (4) | New Kalgen | Polyoxyalkylene allyl | Takemoto | 5 |
| | FS-7 | phenyl ether sulfate | Oil and Fats | |
| | | | Co., Ltd. | |

Compound (1), components (2) and (3) are mixed and passed through a pulverizer, and then component (4) and water are added thereto. The mixture is kneaded and extruded and granulated, followed by drying and size-adjusting to obtain water-dispersible granules.

### FORMULATION EXAMPLE 6

### Water-Based Suspension Concentrate

| | | | | % |
|---|---|---|---|---|
| | Trade Name | Chemical Name | Supplier | wt./wt |
| (1) | Compound of | | | 28 |
| | this invention | | | |
| (2) | Soprophor FL | Triethanolamine | Rhone-Poulenc | 2 |
| | | salts of oxyethlated | | |
| | | polyarylphenol | | |
| | | phosphate | | |
| (3) | Sorpol 355 | A mixture of | Toho Chemical | 1 |
| | | polyoxyethylene | Industry | |
| | | styryl phenyl ether | Co., Ltd. | |
| | | and alkyl aryl | | |
| | | sulfonate | | |
| (4) | IP solvent | Isoparaffin | Idemitsu | 32 |
| | 1620 | hydrocarbon | Petrochemical | |
| | | | Co., Ltd. | |
| (5) | Ethylene glycol | | | 6 |
| (6) | Water | | | 31 |

The compound (1) and the above components (2) to (6) are mixed and ground by a wet-milling machine (Dyno-mill) to obtain a water based suspension concentrate.

The herbicidal properties of the compounds of this invention were discovered in a number of greenhouse tests. The test procedures and results follow.

### TEST EXAMPLE 1

Upland field soil was put into a 1/170,000 ha pot, and seeds of various plants [1. barnyardgrass (Echinochloa crus-galli L.): ECHCC, 2. crabgrass (Digitaria sanguinalis L.): DIGSA, 3. green foxtail (Setaria viridis L.): SETVI, 4. redroot pigweed (Amaranthus retroflexus L.): AMARE, 5. prickly sida (Sida spinosa L.): SIDSP, 6. velvetleaf (Abutilon theophrasti): ABUTH, 7. common cocklebur (Xanthium strumarium L.): XANST, 8. rice (Oryza sativa L.): ORYSA, 9. wheat (Triticum aestivum L.): TRZAX, 10. corn (Zea mays L.): ZEAMX, 11. soybean (Glycine max Merr.): GLXMA] were sown. Then, one day after the sowing, a wettable powder or emulsifiable concentrate having the compound of the present invention formulated in accordance with a usual formulation method, was weighed so that the active ingredient would be a predetermined amount, and diluted with water in an amount of 500 L/ha. The herbicide adjusted was applied by a small size sprayer for pre-emergence treatment. On the 20^{th} to 21^{st} day after the application of the herbicide, the growth of the respective plants was visually observed, and the herbicidal effects were evaluated by growth controlling degrees (%) ranging from 0 (equivalent to the untreated control) to 100 (complete kill), whereby the results shown in Table 11, were obtained. Compound Nos. in Table 11 correspond to Compound Nos. in Table 1 to 7 given hereinbefore.

TABLE 11
Pre-emergence Herbicidal Activity

| Compound | Rate | | | | | | | | | | | |
|---|---|---|---|---|---|---|---|---|---|---|---|---|
| No. | g a.i./ha | ECHCG | DIGSP | SETVI | AMARE | SIDSP | ABUTH | XANST | ORYSA | TRZAX | ZEAMX | GLXMA |
| 1-2 | 125 | 100 | 100 | 100 | 100 | 100 | 100 | 100 | 20 | — | 10 | 10 |
| 1-8 | 250 | 50 | 98 | 95 | 100 | 98 | 100 | 10 | 20 | — | 10 | 10 |
| 1-33 | 125 | 100 | 100 | 100 | 100 | 100 | 100 | 70 | 70 | 70 | 100 | 40 |
| 1-39 | 250 | 100 | 100 | 100 | 100 | 100 | 100 | 100 | 50 | — | 70 | 30 |
| 1-42 | 250 | 100 | 100 | 100 | 100 | 80 | 50 | 0 | 40 | 0 | 10 | 10 |
| 1-79 | 250 | 100 | — | — | 100 | 80 | 60 | — | 30 | 10 | 10 | 30 |
| 1-85 | 250 | 60 | 60 | 80 | 95 | 20 | 30 | 0 | 10 | — | 10 | 0 |
| 1-101 | 125 | 80 | 100 | 100 | 100 | 80 | 60 | 30 | 30 | 20 | 40 | 10 |
| 7-2 | 250 | 100 | 100 | 100 | 100 | 100 | 100 | 50 | 50 | 50 | 60 | 50 |
| 7-4 | 500 | 70 | 100 | 100 | 100 | 100 | 100 | 10 | 30 | — | 40 | — |

### TEST EXAMPLE 2

Upland field soil was put into a 1/170,000 ha pot, and seeds of various plants were sown. Then, when the plants reached predetermined leaf stages [I. barnyardgrass (Echinochloa crus-galli L.), ECHCG: 1.8-2.4 leaf stage, 2. crabgrass (Digitaria sanguinalis L.), DIGSA: 1.5-2.2 leaf stage, 3. green foxtail (Setaria viridis L.), SETVI: 1.7-2.5 leaf stage, 4. redroot pigweed (Amaranthus retroflexus L.), AMARE: 0.1-0.3 leaf stage, 5. prickly sida (Sida spinosa L.), SIDSP: 0.3-0.5 leaf stage, 6. velvetleaf (Abutilon theophrasti), ABUTH: 0.3-0.4 leaf stage, 7. common cocklebur (Xanthium strumarium L.), XANST: 0.2-0.6 leaf stage, 8. rice (Oryza sativa L.), ORYSA: 1.6-2.2 leaf stage, 9. wheat (Triticum aestivum L.), TRZAX: 2.8 leaf stage, 10. corn (Zea mays L.), ZEAMX: 2.2-2.5 leaf stage, 11. soybean (Glycine max Merr.), GLXMA: 0.2-0.3 leaf stage], a wettable powder or emulsifiable concentrate having the compound of the present invention formulated in accordance with a usual formulation method, was weighed so that the active ingredient would be a predetermined amount, and diluted with water in an amount of 500 L/ha. To the diluted solution, 0.1%(v/v) of an agricultural spreader was added. The herbicide adjusted was applied by a small size sprayer for foliage treatment. On the 215′ day after the application of the herbicide, the growth of the respective plants was visually observed, and the herbicidal effects were evaluated by growth controlling degrees (%) ranging from 0 (equivalent to the untreated control) to 100 (complete kill), whereby the results shown in Table 12, were obtained. Compound Nos. in Table 12 correspond to Compound Nos. in Table 1 to 7 given hereinbefore.

TABLE 12
Post-emergence Herbicidal Activity

| Compound | Rate | | | | | | | | | | | |
|---|---|---|---|---|---|---|---|---|---|---|---|---|
| No. | g a.i./ha | ECHCG | DIGSP | SETVI | AMARE | SIDSP | ABUTH | XANST | ORYSA | TRZAX | ZEAMX | GLXMA |
| 1-2 | 63 | 98 | 60 | 70 | 100 | 98 | 100 | 100 | 50 | 30 | 60 | 60 |
| 1-8 | 31 | 0 | 10 | 30 | 95 | 80 | 100 | 70 | 40 | — | 50 | 40 |
| 1-33 | 63 | 100 | 100 | 100 | 100 | 100 | 100 | 100 | 98 | 98 | 100 | 100 |
| 1-39 | 31 | 100 | 100 | 100 | 100 | 100 | 100 | 100 | 98 | — | 98 | 100 |
| 1-40 | 31 | 100 | 100 | 100 | 100 | 100 | 100 | 100 | 70 | — | 70 | 60 |
| 1-42 | 63 | 60 | 50 | 98 | 100 | 100 | 100 | 100 | 70 | — | 30 | 60 |
| 1-79 | 31 | 95 | 40 | 100 | 90 | 98 | 100 | 100 | 50 | — | 50 | 70 |
| 1-85 | 31 | 70 | 40 | 80 | 70 | 95 | 100 | 100 | 40 | — | 50 | 50 |
| 1-101 | 31 | 100 | 100 | 100 | 100 | 100 | 100 | 100 | 60 | 30 | 70 | 70 |
| 7-2 | 63 | 80 | 95 | 100 | — | 100 | 100 | 70 | 60 | — | 90 | 60 |
| 7-4 | 63 | 50 | 50 | 80 | 95 | 80 | 100 | — | 60 | — | 70 | 50 |

### TEST EXAMPLE 3

Paddy field soil was put into a 1/1,000,000 ha pot, and seeds of barnyardgrass (Echinochloa oryzicola: ECHOR) and japanese bulrush (Scirpus juncoides: SCPJO) were sown and slightly covered with soil. Then the pot was left to stand still in a greenhouse in a state where the depth of flooding water was from 0.5 to 1 cm, and one day later, tubers of japanese ribbon wapato (Sagittaria pygmaea: SAGPY) were planted. Thereafter, the depth of flooding water was maintained at a level of from 3 to 4 cm, and when barnyardgrass and japanese bulrush reached a 0.5 leaf stage, an aqueous diluted solution of a wettable powder or emulsifiable concentrate having the compound of the present invention formulated in accordance with a usual formulation method, was uniformly applied under submerged condition by a pipette so that the dose of the active ingredient would be at a predetermined level. On the other hand, paddy filed soil was put into a 1/1,000,000 ha pot and puddled and leveled, and the depth of flooding water was from 3 to 4 cm. One day later, rice (Oryza sativa L.: ORYSA) of 2 leaf stage was transplanted in a depth of 3 cm. On the 4^{th} day after the transplantation, the compound of the present invention was applied in the same manner as described above. On the 14^{th} day after the application of the herbicide, the growth of barnyardgrass, japanese burlush and japanese ribbon wapato was visually observed and on the 21^{st} day after the application of the herbicide, the growth of rice was visually observed, and the herbicidal effects were evaluated by growth controlling degrees (%) ranging from 0 (equivalent to the untreated control) to 100 (complete kill), whereby the results shown in Table 13, were obtained. Compound Nos. in Table 13 correspond to Compound Nos. in Table 1 to 7 given hereinbefore.

TABLE 13
Water Application Herbicidal Activity

| Compound | Rate | | | | |
|---|---|---|---|---|---|
| No. | g a.i./ha | ECHOR | SCPJO | SAGPY | ORYSA |
| 1-2 | 63 | 40 | 30 | 0 | 20 |
| 1-8 | 63 | 60 | 60 | 40 | 10 |
| 1-33 | 63 | 100 | 100 | 100 | 80 |
| 1-39 | 63 | 100 | 100 | 100 | 60 |
| 1-40 | 63 | 100 | 100 | 95 | 35 |
| 1-42 | 63 | 100 | 98 | 100 | 35 |
| 1-79 | 63 | 100 | 100 | 100 | 40 |
| 1-85 | 63 | 100 | 98 | 100 | 10 |
| 1-101 | 63 | 100 | 50 | 100 | 35 |
| 7-2 | 63 | 100 | 98 | — | 0 |
| 7-4 | 63 | 70 | 90 | — | 0 |

Variations of the invention will be apparent to the skilled artisan from the above description.

What we claimed is:

1. A compound of the formula I or its salt: 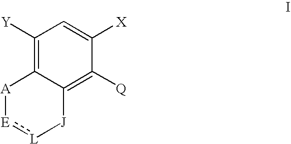
wherein X and Y are independent of each other and are hydrogen, halogen, cyano, nitro, (C₁₋₄)alkyl, (C₁₋₄)alkoxy, (C₁₋₄)haloalkyl or (C₁₋₄)haloalkoxy;
A is oxygen or CR₁R₂; E and L are independent of each other and are selected from CR₃, CR₃R₄, oxygen, nitrogen, NR₅, S(O)n, C(═O), C(═S), or C(═NR₆); J is oxygen or CR₇R₈; when A is CR₁R₂, J is oxygen; R₁, R₂, R₃, R₄, R₅, R₆, R₇ and R₈ are independent of each other and are selected from the group consisting of hydrogen, halogen, cyano; nitro, amino, hydroxy; mercapto, carboxyl; (C₁₋₁₂)alkyl, (C₁₋₆)alkoxy, (C₂₋₆)alkenyl, (C₂₋₆)alkynyl, (C₂₋₆)alkenyloxy, (C₂₋₆)alkynyloxy, aryl, heteroaryl, aryloxy, heteroaryloxy, (C₃₋₆)cycloalkyl, (C₃₋₆)cycloalkylcarbonyl, (C₁₋₆)alkylcarbonyl, arylcarbonyl, di(C₁₋₆alkyl)aminocarbonyl, (C₁₋₆)alkylcarbonyloxy, (C₁₋₆)alkoxycarbonyl, (C₁₋₆)alkylthiocarbonyl, (C₁₋₆)alkoxythiocarbonyl, (C₁₋₆)alkylthio, arylthio, (C₂₋₆)alkenylthio, (C₂₋₆)alkynylthio, (C₁₋₆)alkylsulfinyl, (C₂₋₆)alkenylsulfinyl, (C₂₋₆)alkynylsulfinyl, (C₁₋₆)alkylsulfonyl, (C₂₋₆)alkenylsulfonyl, (C₂₋₆)alkynylsulfonyl, arylsulfinyl, arylsulfonyl, —CR₁₃═NOR₁₄ and —CO₂NR₁₃R₁₄, where any of these groups may be substituted with one or more of the following group consisting of halogen, hydroxy, mercapto, cyano, nitro, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkylsulfonyloxy, (C₁₋₆)haloalkylsulfonyloxy, (C₁₋₆)alkoxy, (C₁₋₆)alkoxycarbonyl, aminocarbonyl, (C₁₋₆)alkylaminocarbonyl, di(C₁₋₆alkyl)aminocarbonyl, arylcarbonyl, haloarylcarbonyl, (C₁₋₆)haloalkoxy, (C₁₋₆)haloalkoxycarbonyl, (C₁₋₆)alkylsulfonyl, (C₁₋₆)haloalkylsulfonyl, arylsulfonyl, haloarylsulfonyl, aryl, haloaryl, alkoxyaryl, aryloxy, arylthio, haloaryloxy, heteroaryl, heteroaryloxy and (C₃₋₇)cycloalkyl; when R₃ and R₄ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, hydroxy, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, aryl, heteroaryl and (C₃₋₇)cycloalkyl; n is represent an integer from 0 to 2;
Q is selected from; 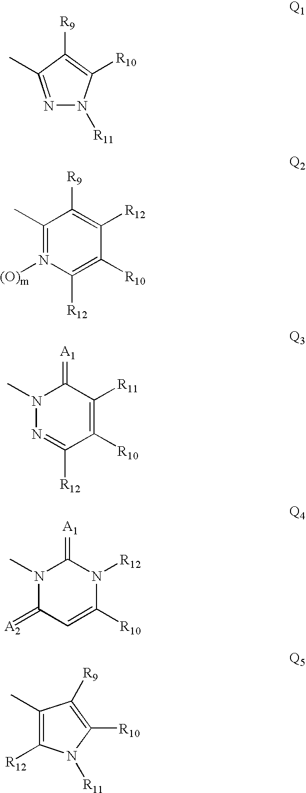
wherein A₁ and A₂ are independently oxygen or sulfur; R₉ and R₁₀ are independent of each other and are selected from the group consisting of halogen, cyano, nitro, formyl, hydroxy, amino, mercapto, (C₁₋₄)alkyl, (C₁₋₄)haloalkyl, (C₁₋₄)alkoxy, (C₁₋₄)haloalkoxy, (C₁₋₄)alkylamino, di(C₁₋₄alkyl)amino, (C₁₋₄)haloalkylamino, di(C₁₋₄haloalkyl)amino, (C₁₋₄)alkoxyamino, di(C₁₋₄alkoxy)amino, (C₁₋₄)haloalkoxyamino, di(C₁₋₄haloalkoxy)amino, (C₁₋₄)alkylcarbonyl, (C₁₋₄)haloalkylcarbonyl, (C₁₋₄)alkoxycarbonyl, (C₁₋₄)haloalkoxycarbonyl, (C₁₋₄)alkylcabonylamino, (C₁₋₄)haloalkylcarbonylamino, (C₁₋₄)alkoxycarbonylamino, (C₁₋₄)haloalkoxycarbonylamino, (C₁₋₆)alkoxyalkyl, (C₁₋₆)haloalkoxyalkyl, (C₁₋₆)alkoxyalkoxy, (C₁₋₆)alkylthio, (C₁₋₆)haloalkylthio, (C₂₋₆)alkenylthio, (C₂₋₆)haloalkenylthio, (C₂₋₆)alkynylthio, (C₂₋₆)haloalkynylthio, (C₁₋₆)alkylsulfinyl, (C₁₋₆)haloalkylsulfinyl, (C₂₋₆)alkenylsulfinyl, (C₂₋₆)haloalkenylsulfinyl, (C₂₋₆)alkynylsulfinyl, (C₂₋₆)haloalkynylsulfinyl, (C₁₋₆)alkylsulfonyl, (C₁₋₆)haloalkylsulfonyl, (C₂₋₆)alkenylsulfonyl, (C₂₋₆)haloalkenylsulfonyl, (C₂₋₆)alkynylsulfonyl, (C₂₋₆)haloalkynylsulfonyl, arylsulfonyl, (C₂₋₆)alkenyl, (C₂₋₆)haloalkenyl, (C₂₋₆)alkynyl and (C₂₋₆)haloalkynyl; R₁₁ is selected from the group consisting of hydrogen, halogen, (C₁₋₃)alkyl, (C₁₋₃)haloalkyl, hydroxy, (C₁₋₃)alkoxy, (C₁₋₃)haloalkoxy, cyano, nitro, amino, (C₁₋₃)alkylcarbonyl, (C₁₋₃)alkoxycarbonyl and (C₁₋₆)alkylamino; R₁₂ is selected from the group consisting of hydrogen, halogen, cyano, amino, (C₁₋₄)alkyl, (C₁₋₄)haloalkyl, (C₁₋₄)alkoxy, (C₁₋₄)haloalkoxy, (C₂₋₆)alkenyl, (C₂₋₆)haloalkenyl and hydroxy; when R₉ and R₁₀ or R₁₀ and R₁₁ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, hydroxy, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, aryl, heteroaryl and (C₃₋₇)cycloalkyl; m is 0 or 1; R₁₃ and R₁₄ are independent of each other and are selected from the group consisting of hydrogen, (C₁₋₆)alkyl and aryl; provided that A is oxygen and J is CR₇R₈, when Q is Q₄.

2. A compound according to claim 1, wherein X and Y are independent of each other and are hydrogen, halogen or cyano; A is oxygen; E and L are independent of each other and are selected from CR₃, CR₃R₄, oxygen, nitrogen, NR₅, S(O)n or C(═O); J is CR₇R₈; R₃, R₄, R₅, R₇ and R₈ are independent of each other and are selected from the group consisting of hydrogen, halogen, cyano, nitro, amino, hydroxy, (C₁₋₆)alkyl, (C₁₋₆)alkoxy, (C₂₋₆)alkenyl, (C₂₋₆)alkynyl, aryl, aryloxy, (C₃₋₆)cycloalkyl, (C₁₋₆)alkylcarbonyl, arylcarbonyl, (C₁₋₆)alkylcarbonyloxy, (C₁₋₆)alkoxycarbonyl, (C₁₋₆)alkylthiocarbonyl, (C₁₋₆)alkoxythiocarbonyl, (C₁₋₆)alkylthio, arylthio, (C₂₋₆)alkenylthio, (C₂₋₆)alkynylthio, (C₁₋₆)alkylsulfinyl, (C₂₋₆)alkenylsulfinyl, (C₂₋₆)alkynylsulfinyl, (C₁₋₆)alkylsulfonyl, (C₂₋₆)alkenylsulfonyl, (C₂₋₆)alkynylsulfonyl, arylsulfinyl and arylsulfonyl, where any of these groups may be substituted with one or more of the following group consisting of halogen, hydroxy, mercapto, cyano, nitro, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, arylsulfonyl, aryl, haloaryl, alkoxyaryl, aryloxy, arylthio, haloaryloxy, heteroaryl, heteroaryloxy and (C₃₋₇)cycloalkyl; n is represent an integer from 0 to 2; Q is Q., Q₂ or Q₅; R₉ and R₁₀ are independent of each other and are selected from the group consisting of halogen, cyano, formyl, hydroxy, amino, mercapto, (C₁₋₄)alkyl, (C₁₋₄)haloalkyl, (C₁₋₄)alkoxy, (C₁₋₄)haloalkoxy, (C₁₋₄)alkylamino, (C₁₋₄)haloalkylamino, (C₁₋₄)alkoxyamino, (C₁₋₄)haloalkoxyamino, (C₁₋₄)alkylcarbonyl, (C₁₋₄)haloalkylcarbonyl, (C₁₋₄)alkoxycarbonyl, (C₁₋₄)haloalkoxycarbonyl, (C₁₋₄)alkylcabonylamino, (C₁₋₄)haloalkylcarbonylamino, (C₁₋₄)alkoxycarbonylamino, (C₁₋₄)haloalkoxycarbonylamino, (C₁₋₆)alkoxyalkyl, (C₁₋₆)haloalkoxyalkyl, (C₁₋₆)alkylthio, (C₁₋₆)haloalkylthio, (C₂₋₆)alkenylthio, (C₂₋₆)haloalkenylthio, (C₂₋₆)alkynylthio, (C₂₋₆)haloalkynylthio, (C₁₋₆)alkylsulfinyl, (C₁₋₆)haloalkylsulfinyl, (C₂₋₆)alkenylsulfinyl, (C₂₋₆)haloalkenylsulfinyl, (C₂₋₆)alkynylsulfinyl, (C₂₋₆)haloalkynylsulfinyl, (C₁₋₆)alkylsulfonyl, (C₁₋₆)haloalkylsulfonyl, (C₂₋₆)alkenylsulfonyl, (C₂₋₆)haloalkenylsulfonyl, (C₂₋₆)alkynylsulfonyl, (C₂₋₆)haloalkynylsulfonyl, arylsulfonyl, (C₂₋₆)alkenyl, (C₂₋₆)haloalkenyl, (C₂₋₆)alkynyl and (C₂₋₆)haloalkynyl; R₁, is selected from the group consisting of hydrogen, (C₁₋₃)alkyl, (C₁₋₃)haloalkyl, (C₁₋₃)alkylcarbonyl and (C₁₋₃)alkoxycarbonyl; R₁₂ is selected from the group consisting of hydrogen, halogen, cyano, (C₁₋₄)alkyl, (C₁₋₄)haloalkyl, (C₁₋₄)alkoxy, (C₁₋₄)haloalkoxy, (C₂₋₆)alkenyl, (C₂₋₆)haloalkenyl and hydroxy; when R₉ and R₁₀ or R₁₀ and R₁₁, are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, hydroxy, amino, carboxyl, (C₁₋₆)alkyl, (C₁₋₆)haloalkyl, (C₁₋₆)alkylcarbonyl, (C₁₋₆)alkylcarbonyloxy, (C₁₋₆)haloalkylcarbonyl, (C₁₋₆)haloalkylcarbonyloxy, (C₁₋₆)alkoxy, (C₁₋₆)alkoxycarbonyl, aminocarbonyl, (C₁₋₆)alkylaminocarbonyl, (C₁₋₆)haloalkoxy, (C₁₋₆)haloalkoxycarbonyl, (C₁₋₆)alkylsulfonyl, (C₁₋₆)haloalkylsulfonyl, aryl, heteroaryl and (C₃₋₇)cycloalkyl.

3. A compound according to claim 1, wherein X and Y are independent of each other and are hydrogen, halogen or cyano; A is oxygen; E and L, respectively, are CR₃R₄ and CR₃′R₄′; J is CR₇R₈; R₃, R₄, R₃′, R₄′, R₇ and R₈ are independent of each other and are selected from the group consisting of hydrogen, halogen, cyano, nitro, hydroxy, (C₁₋₆)alkyl, (C₁₋₆)haloalkyl, (C₁₋₆)alkoxy, (C₁₋₆)haloalkoxy, (C₁₋₆)alkoxyalkyl, (C₂₋₆)alkenyl, (C₂₋₆)alkynyl, aryl, aryloxy, (C₃₋₆)cycloalkyl, (C₁₋₆)alkylcarbonyl, arylcarbonyl, (C₁₋₃)haloalkylcarbonyl, (C₁₋₆)alkylcarbonyloxy, (C₁₋₆)haloalkylcarbonyloxy, (C₁₋₆)alkoxycarbonyl, (C₁₋₆)haloalkoxycarbonyl, (C₁₋₆)alkylthiocarbonyl, (C₁₋₆)haloalkylthiocarbonyl, (C₁₋₆)alkoxythiocarbonyl, (C₁₋₆)haloalkoxythiocarbonyl, arylsulfonylamino, arylamino, (C₁₋₆)alkylthio, arylthio, (C₂₋₆)alkenylthio, (C₂₋₆)alkynylthio, (C₁₋₆)alkylsulfinyl, (C₂₋₆)alkenylsulfinyl, (C₂₋₆)alkynylsulfinyl, (C₁₋₆)alkylsulfonyl, (C₂₋₆)alkenylsulfonyl, (C₂₋₆)alkynylsulfonyl, arylsulfinyl and arylsulfonyl; Q is Q₁ or Q₂; R₉ and R₁₀ are independent of each other and are selected from the group consisting of halogen, cyano, hydroxy, amino, (C₁₋₄)alkyl, (C₁₋₄)haloalkyl, (C₁₋₄)alkoxy and (C₁₋₄)haloalkoxy; R₁₁ is selected from the group consisting of hydrogen, (C₁₋₃)alkyl and (C₁₋₃)haloalkyl; R₁₂ is selected from the group consisting of hydrogen, halogen, cyano, (C₁₋₄)alkyl, (C₁₋₄)haloalkyl, (C₁₋₄)alkoxy and (C₁₋₄)haloalkoxy; when R₉ and R₁₀ or R₁₀ and R₁₁, are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, (C₁₋₆)alkyl, (C₁₋₆)haloalkyl and (C₁₋₆)alkoxy; m is 0; n is represent an integer from 0 to 2.

4. A compound according to claim 1, wherein X and Y are independent of each other and are hydrogen or halogen; A is oxygen; E and L, respectively, are CR₃R₄ and CR₃′R₄′; J is CH₂; R₃, R₄, R₃′, and R₄′ are independent of each other and are selected from the group consisting of hydrogen, halogen, cyano, (C₁₋₆)alkyl, (C₁₋₆)haloalkyl, (C₁₋₆)alkoxy, (C₁₋₆)haloalkoxy, (C₁₋₆)alkoxyalkyl, (C₂₋₆)alkenyl, aryl, aryloxy, (C₃₋₆)cycloalkyl, (C₁₋₆)alkylcarbonyl, (C₁₋₃)haloalkylcarbonyl, (C₁₋₆)alkylcarbonyloxy, (C₁₋₆)haloalkylcarbonyloxy, (C₁₋₆)alkoxycarbonyl, (C₁₋₆)alkylthiocarbonyl, (C₁₋₆)alkoxythiocarbonyl, (C₁₋₆)haloalkoxythiocarbonyl, (C₁₋₆)alkylthio, arylthio, (C₂₋₆)alkynylthio, (C₁₋₆)alkylsulfinyl and (C₁₋₆)alkylsulfonyl; Q is Q₁ or Q₂; R₉ and R₁₀ are independent of each other and are selected from the group consisting of halogen, (C₁₋₄)alkyl, (C₁₋₄)haloalkyl and (C₁₋₄)haloalkoxy; R₁₁ is (C₁₋₃)alkyl; R₁₂ is selected from the group consisting of hydrogen and halogen; when R₉ and R₁₀ or R₁₀ and R₁₁ are taken together with the atoms to which they are attached, they represent a three to seven membered substituted or unsubstituted ring optionally containing oxygen, S(O)n or nitrogen with following optional substitutions, one to three substituents selected from the group consisting of halogen, cyano, nitro, (C₁₋₆)alkyl, (C₁₋₆)haloalkyl and (C₁₋₆)alkoxy; m is 0; n is represent an integer from 0 to 2.

5. A process for the preparation of the intermediate of the formula XVII, which comprises reducing a compound according to formula XV: 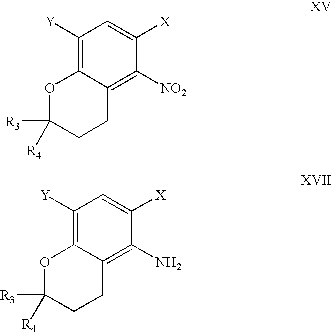
wherein X, Y, R₃ and R₄ are as defined in claim 1.

6. A process for the preparation of the intermediate of the formula XVII, which comprises reducing a compound according to formula XXXVIII: 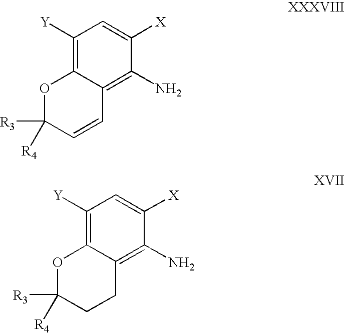
wherein X, Y, R₃ and R₄ are as defined in claim 1.

7. A process for the preparation of the intermediate of the formula XXII, which comprises oxidizing a compound according to formula XXI: 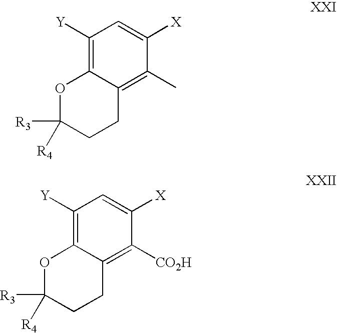
wherein X, Y, R₃ and R₄ are as defined in claim 1.

8. A process for the preparation of the intermediate of the formula XXII, which comprises carboxylating a compound according to formula XXXIII: 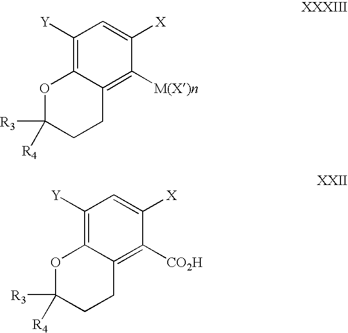
wherein M is lithium, magnesium, zinc, tin or boron; X′ is halogen, (C₁₋₆)alkyl or hydroxy; n is 0, 1, 2, 3 or 4; and X, Y, R₃ and R₄ are as defined in claim 1.

9. A process for the preparation of the intermediate of the formula XXXII, which comprises reducing a compound according to formula XXXI: 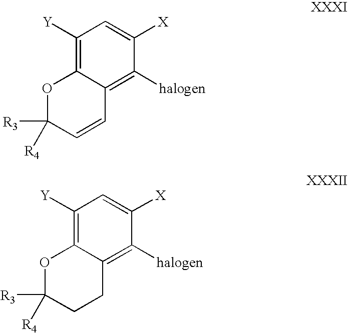
wherein X, Y, R₃ and R₄ are as defined in claim 1.

10. A process for the preparation of the compound of formula VI, which comprises reducing a compound according to formula V: 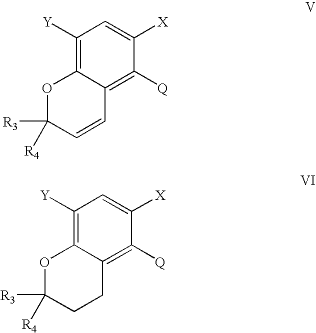
wherein X, Y, R₃, R₄ and Q are as defined in claim 1.

11. A process for the preparation of the compound of the formula VI, which comprises ring-forming a compound according to formula II′ with olefinic compound or alcohol derivative: 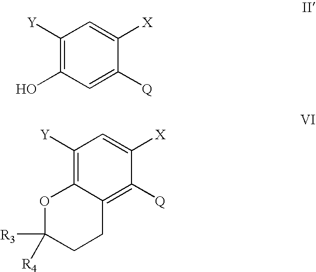
wherein X, Y, R₃, R₄ and Q are as defined in claim 1.

12. A process for the preparation of the intermediate of formula XXXXVII, which comprises derivatizing a compound according to formula XIV with R″COCl: 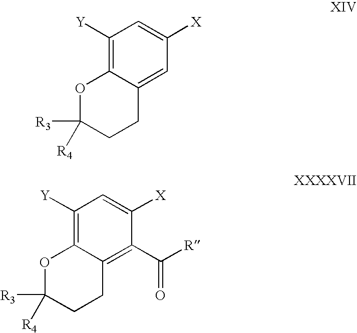
wherein R″ is (C₁₋₆)alkyl; and X, Y, R₃ and R₄ are as defined in claim 1.

13. A herbicidal composition comprising at least one compound according to claim 1 and an agricultural additive.

14. A defoliate composition comprising at least one compound according to claim 1 and an agricultural additive.

15. A herbicidal composition comprising at least one member selected from a compound according to claim 1 and at least one other herbicide.

16. A method for controlling undesired vegetation in a crop field by applying to the locus of the crop to be protected a herbicidally effective amount of a compound according to claim 1.

17. The method according to claim 16, wherein the crop field is corn, soybean, wheat or rice field.

18. A method to defoliate potato and cotton using a compound according to claim 1.

19. The method according to claim 16, wherein the compound of claim 11 is applied as a pre-emergent herbicide.

20. The method according to claim 16, wherein the compound of claim 11 is applied as a post-emergent herbicide.
